(19) United States
(12) Patent Application Publication
(10) Pub. No.: US 2016/0185735 A1
(43) Pub. Date: Jun. 30, 2016

(54) FATTY ACID ANALOGS AND THEIR USE

(71) Applicant: UNIVERSITY OF PITTSBURGH - OF THE COMMONWEALTH SYSTEM OF HIGHER EDUCATION, Pittsburgh, PA (US)

(72) Inventors: Carolyn Anderson, Pittsburgh, PA (US); Zhengxin Cai, Swissvale, PA (US); Bennett Van Houten, Pittsburgh, PA (US); Wilson B Edwards, Pittsburgh, PA (US)

(73) Assignee: University of Pittsburgh - Of the Commonwealth System of Higher Education, Pittsburgh, PA (US)

(21) Appl. No.: 14/911,457

(22) PCT Filed: Aug. 12, 2014
(86) PCT No.: PCT/US14/50643

§ 371 Date: Feb. 10, 2016

Related U.S. Application Data
(60) Provisional application No. 61/865,067, filed on Aug. 12, 2013.

(51) Int. Cl. C07D 249/04; A61K 51/04; G01N 33/52

(52) U.S. Cl. CPC C07D 249/04; G01N 33/52; A61K 51/0402

(57) ABSTRACT

Embodiments of novel fatty acid analogs, precursors of such analogs, pharmaceutical compositions including same, and methods of their use are disclosed. In several embodiments, the disclosed fatty acid analogs can be used to detect and monitor β-oxidation in cells and tissue, for example, in methods of detecting a tumor, such as a prostate tumor.

### CROSS REFERENCE TO RELATED APPLICATIONS

This application claims priority to U.S. Provisional Application No. 61/865,067, filed Aug. 12, 2013, which is incorporated by reference in its entirety.

### FIELD

This relates to labeled fatty acid analogs, and to methods of making and using the same.

### BACKGROUND

Some tumor types and other diseases, such as cardiovascular disease and diabetes, are known to have upregulated or abnormal fatty acid uptake and consumption. Positron emission tomography (PET) imaging of fatty acid metabolism can provide a noninvasive means of diagnosing primary tumors, such as prostate cancer, staging and re-staging patient populations, classifying prostate cancer based on its metabolism, monitoring therapeutic effects, and early detection of metastasis or other diseases. The information gained from imaging β-oxidation of fatty acids in tumors provides the clinician with important information for diagnosis, prognosis, and treatment decisions.

### SUMMARY

Novel fatty acid analogs are disclosed herein. In several embodiments, the analogs include a detectable moiety, a triazole ring and a sulfur atom substitution in the fatty acid carbon backbone. The disclosed analogs have improved pharmacokinetics and radiolabeling characteristics compared to known compounds. The analogs are useful, for example, as PET tracers to identify cells with increased β-oxidation of fatty acids (e.g., tumor cells).

In some embodiments, a fatty acid analog or salt thereof is provided comprising a structure according to general formula I or II:

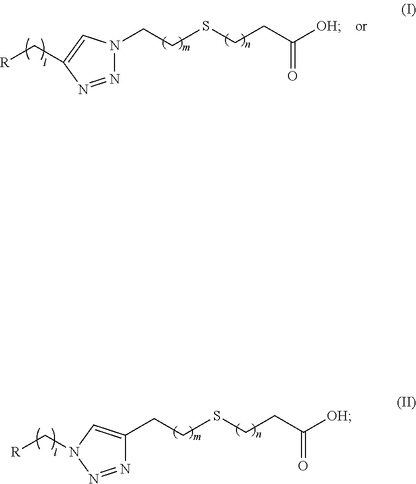

wherein R is a detectable moiety, and wherein l, m, and n independently are from 0 to 20, and wherein the carbon backbone of the compound optionally comprises one or more unsaturated double or triple bonds. In an alternative embodiment, a fatty acid analog or salt thereof is provided comprising a structure according to general formula I or II, wherein R is a detectable moiety, and wherein the sum of l, m, and n is from 0 to 22, and wherein the carbon backbone of the compound optionally comprises one or more unsaturated double or triple bonds. In some embodiments, the detectable moiety can be a radiolabeled halogen atom, such as ⁸F, or a fluorescent moiety, such as Cy3. In more embodiments, the fatty acid analog can be a palmitate or oleate analog, for example, comprising a structure according to one of formulas (III)-(VI), or a pharmaceutically acceptable salt thereof:

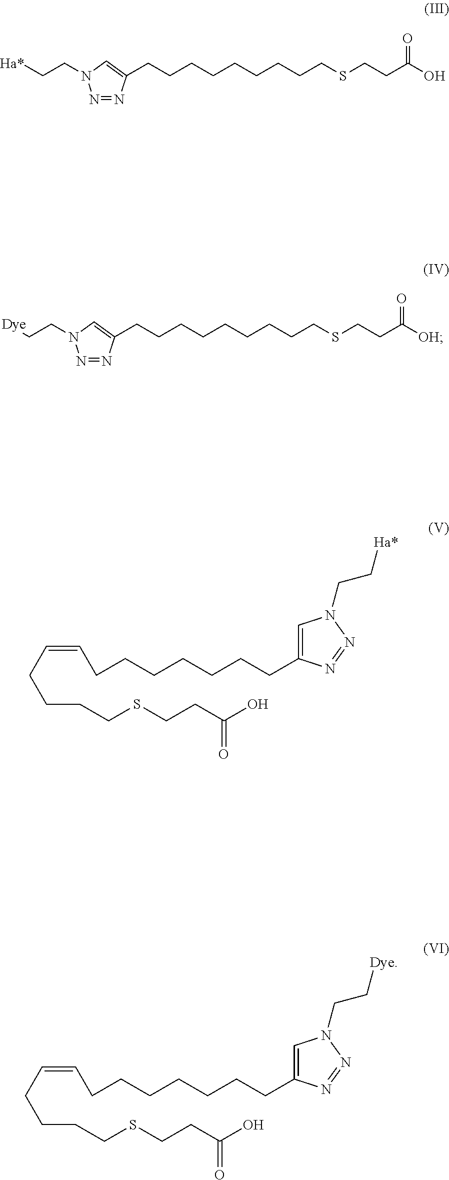

wherein HA* is a radioactive halogen and Dye is a visible marker such as a fluorophore or chromophore.

Compositions comprising the disclosed fatty acid analogs, and methods of their use also provided. In some embodiments, the method comprises contacting a cell with an effective amount of a composition comprising a disclosed fatty acid analog comprising a detectable moiety under conditions sufficient for β-oxidation of the fatty acid analog in the cell, and detecting the detectable moiety on the fatty acid analog in the cell.

In some embodiments, a method is provided comprising contacting a cell with an effective amount of a fatty acid analog or salt thereof comprising a structure according to general formula XI:

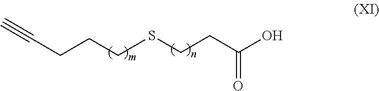

under conditions sufficient for β-oxidation of the fatty acid analog in the cell, wherein m and n independently are from 0 to 20, and wherein the carbon backbone of the compound optionally comprises one or more unsaturated double or triple bonds. In an alternative embodiment, a method is provided comprising contacting a cell with an effective amount of a fatty acid analog or salt thereof comprising a structure according to general formula XI, under conditions sufficient for β-oxidation of the fatty acid analog in the cell, wherein the sum of m and n is from 0 to 22, and wherein the carbon backbone of the compound optionally comprises one or more unsaturated double or triple bonds. The method further includes contacting the cell with an effective amount of a detectable moiety linked to an azide functional group under conditions sufficient for 1,3-dipolar cycloaddition, and detecting the detectable moiety linked to the fatty acid analog in the cell. The method is useful, for example, for detecting increased β-oxidation of fatty acids in a cell or tissue (such as a tumor cell or tissue).

In more embodiments, a method of detecting a cell or tissue in a subject with increased β-oxidation of fatty acids is provided, comprising administering an effective amount of a disclosed fatty acid analog or salt thereof to the subject; and detecting an increase in the presence of the detectable moiety in the cell or tissue of the subject compared to a control; thereby detecting the cell or tissue in the subject with increased β-oxidation of fatty acids. In some embodiments, the cell or tissue can be a tumor cell or tissue, such as a prostate cancer tumor cell or tissue.

The foregoing and other features and advantages of this disclosure will become more apparent from the following detailed description of several embodiments which proceeds with reference to the accompanying figures.

### BRIEF DESCRIPTION OF THE FIGURES

FIG. 1 is a diagram illustrating the chemical structure of [¹⁸F]Fluoro-4-thia-oleic acid (FTO).

FIG. 2 is a series of diagrams illustrating the synthesis of precursors of labeled oleate analogs that can be used for dye-labeling and ¹⁸F-labeling of the oleate analog.

FIG. 3 is a set of diagrams illustrating synthesis of ¹⁸F-labeled oleate analog.

FIG. 4 is a series of diagrams illustrating synthesis of Cy3 labeled oleate analog.

FIG. 5 is a series of diagrams illustrating synthesis of precursors of labeled palmitate analogs that can be used for dye-labeling and ¹⁸F-labeling of the palmitate analog.

FIG. 6 is a set of diagrams illustrating synthesis of the ¹⁸F-labeled palmitate analog.

FIG. 7 is a set of diagrams illustrating synthesis of Cy3-labeled palmitate analog.

FIG. 8 shows a set of immunofluorescence images illustrating cell uptake and co-localization of the Cy3-labeled oleate analog (compound XX) with mitochondria in the PC3 cell line. Top-left shows the Cy3 fluorescence of the fatty acid analog; top-right shows the fluorescence of labeled mitochondria. Bottom figures show the merged images indicating co-localization of the fatty acid analog with mitochondria.

FIGS. 9-10 are graphs showing the distribution of ¹⁸F signal in heart tissue taken from fasted mice (ICR strain) administered the ¹⁸F-labeled oleate analog (compound Va). Folch-type extraction was used to separate “Organic” (unoxidized analog) from “Pellets” and “Aqueous” (oxidized fatty acid metabolites). Over time, more of the fatty acid analog was oxidized. Etomoxir, a known inhibitor of β-oxidation, reduced the extent of the β-oxidation of the ¹⁸F-labeled oleate analog in heart tissue. **: P<0.05; ns: no significant difference.

FIGS. 11 and 12 are graphs showing biodistribution of ¹⁸F-labeled FTO (“FTO,” left bar, Compound I) and the ¹⁸F-labeled oleate analog (“clicked FTO,” right bar, compound Va) at 0.5 hours (FIG. 11) and 2 hours (FIG. 12) postinjection into mice (ICR strain). Similar to ¹⁸F-FTO, the ¹⁸F-labeled oleate analog has low background muscle uptake at 0.5 and 2 hours postinjection. The myocardial uptake of the ¹⁸F-labeled oleate analog is lower than the ¹⁸F-FTO, but the ¹⁸F-labeled oleate analog has significantly less in vivo defluorination than the ¹⁸F-FTO at 0.5 hours postinjection. Due to the introduction of the triazole group, in vivo defluorination of the clicked FTO was significantly inhibited relative to the FTO at 2 hours postinjection.

FIG. 13 is a graph showing the blockage of β-oxidation of ¹⁸F-labeled FTO (“FTO,” left bar, Compound I) and the ¹⁸F-labeled oleate analog (“clicked FTO,” right bar, compound Va) with etomoxir. The myocardial uptake of the clicked FTO can be blocked with etomoxir, indicating that part of the PET signal reflects the metabolism of the fatty acid.

FIG. 14 is a table showing tissue biodistribution of ¹⁸F-labeled-Oleate analog (compound Va) in mice (ICR strain).

FIGS. 15-16 are transaxial PET images of heart tissue of fasted mice (ICR strain) treated with ¹⁸F-labeled palmitate analog (Compound IIIa; FIG. 15) and ¹⁸F-labeled oleate analog (Compound Va; FIG. 16).

FIG. 17 shows PET images of ¹⁸F-labeled oleate analog (compound Va) uptake in PC3 xenografts. The upper row shows PET images of mice administered the ¹⁸F-labeled oleate analog after inhibiting glycolysis with FDG and 2-DG. The lower row shows PET images of mice from the control group without FDG and 2-DG treatment.

FIG. 18 shows a set of fluorescence microscopic images of fatty acids analogs in PC3 cells post-fixation. Cells were treated with Alkyn-4-Thia-Palmitate, Alkyn-Palmitate, or control. The cells were then fixed, and incubated with sulfo-Cy3 azide to detect the trapped fatty acid analog.

### DETAILED DESCRIPTION

This disclosure is directed toward novel fatty acid analogs that undergo β-oxidation in cells, and methods of their use for imaging tumor cells or other diseases or conditions where monitoring of fatty acid metabolism is useful. In several embodiments, the fatty acid analogs are designed for use as PET and optical imaging agents. PET imaging is used in a variety of medical applications, including imaging of tumors, the cardiovascular system, and the brain. Optical imaging is mainly used for preclinical studies at cellular or tissue levels; however it is also used clinically as a diagnostic tool or used to assist surgery. The fatty acid analogs also can be utilized for intraoperative imaging of tumors or imaging tumors that are closer to the surface, or otherwise accessible by imaging probes in various cavities (e.g. oral, etc.).

[¹⁸F]Fluoro-4-thia-oleic acid (FTO) is a known PET tracer that undergoes β-oxidation in the mitochondria. Following β-oxidation, FTO is trapped intracellularly making it useful for monitoring fatty acid metabolism, and for imaging of cells and tissue with high levels of fatty acid metabolism, such as imaging of cardiac tissue. The disclosed fatty acid analogs include a combination of features that provides superior properties for use in methods of detecting and/or monitoring fatty acid metabolism in a cell or tissue compared to known PET tracers, such as FTO.

For example, the disclosed fatty acid analogs include a sulfur atom substitution in the carbon backbone, which allows the intracellular trapping of the fatty acid analog, once it is oxidized through β-oxidation. Further, the disclosed analogs include a triazole group for increased hydrophilicity through formation of intra- and inter-molecular hydrogen bonds. These structural changes reduce unwanted incorporation of the disclosed analogs into lipid bilayers. Further, the design of the disclosed fatty acid analogs allows use of precursor compounds including azide or alkyne group for improved labeling functionality (e.g., with click chemistry, which allows the easy attachment of almost any moiety, halogen, dye or other like chelators). Taken together, these features provide unexpectedly improved compounds for the monitoring of fatty acid metabolism.

### I. SUMMARY OF TERMS

Unless otherwise noted, technical terms are used according to conventional usage. Definitions of common terms in chemistry may be found in Richard J. Lewis, Sr. (ed.), Hawley's Condensed Chemical Dictionary, published by John Wiley & Sons, Inc., 1997 (ISBN 0-471-29205-2). Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

As used herein, the singular forms “a,” “an,” and “the,” refer to both the singular as well as plural, unless the context clearly indicates otherwise. The phrase “and/or” means “and” or “or.” In order to facilitate review of the various embodiments, unless context indicates otherwise, all numbers expressing quantities of reactants and products, properties such as molecular weight, percentages, dosages, and so forth, as used in the specification or claims are to be understood as being modified by the term “about.” Accordingly, unless otherwise indicated, implicitly or explicitly, the numerical parameters and/or non-numerical properties set forth are approximations that may depend on the desired properties sought, limits of detection under standard test conditions/methods, limitations of the processing method, and/or the nature of the parameter or property. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

β-Oxidation: The process by which fatty acid molecules are broken down in the mitochondria to generate acetyl coenzyme A. Methods of detecting β-oxidation in a cell or in a subject are provided herein.

Administration: To provide or giving to a subject an agent, for example, a composition that includes a fatty acid analog, by any effective route. Exemplary routes of administration include, but are not limited to, oral, parenteral (for example, intramuscular, intraperitoneal, intravenous, ICV, intracisternal injection or infusion, subcutaneous injection, or implant), by inhalation spray, nasal, vaginal, rectal, sublingual, urethral (for example, urethral suppository) or topical routes of administration (for example, gel, ointment, cream, aerosol, etc.). The fatty acid analog or salt thereof may be formulated, alone or together, in suitable dosage unit formulations containing conventional non-toxic pharmaceutically acceptable carriers, adjuvants, excipients and vehicles appropriate for each route of administration. In addition to administration to warm-blooded animals such as mice, rats, horses, cattle, sheep, dogs, cats, monkeys, etc., the compounds described herein may be administered to humans.

Analog, Derivative or Mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Biological sample: A sample obtained from a subject. Biological samples include all clinical samples useful for detection of disease or infection (for example, cancer) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having a tumor, such as a prostate cancer tumor.

Click Chemistry: A type of chemical reaction tailored to generate covalent bonds quickly and reliably by joining small units comprising reactive groups together. In general, click chemistry reactions require at least two molecules, each comprising a click chemistry functional group that can react with each other. A click chemistry functional group is a reactive group that can partake in a click chemistry reaction. A non-limiting example of a click chemistry reaction is the alkyne-azide cycloaddition reaction, which involves a 1,3-dipolar cycloaddition reaction between an azide functional group on a first reactant and an alkyne functional group on a second reactant. The 1,3-dipolar cycloaddition reaction is typically performed in the presence of catalyst (e.g., copper) to form the triazole containing product. Exemplary click chemistry functional groups suitable for use according to some aspects of this invention are described herein. Other suitable click chemistry functional groups are known to those of skill in the art (see, e.g., Lahann (Ed), Click Chemistry for Biotechnology and Materials Science. Wiley, 2009, incorporated by reference herein in its entirety).

Contacting: Placement in direct physical association, for example solid, liquid or gaseous forms. Contacting includes, for example, direct physical association of fully- and partially-solvated molecules.

Control: A reference standard. In some embodiments, the control is a negative control, such as sample obtained from a healthy patient without a tumor. In other embodiments, the control is a positive control, such as a tissue sample obtained from a patient with a tumor. In still other embodiments, the control is a historical control or standard reference value or range of values. A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Detectable Moiety: a moiety that is conjugated directly or indirectly to a second molecule, such as a fatty acid analog, to facilitate detection of the second molecule. For example, the detectable moiety can be capable of detection by microscopy or diagnostic imaging techniques (such as PET scans, CT scans, MRIs, ultrasound, fiberoptic examination, and laparoscopic examination). Specific, non-limiting examples of detectable moieties include fluorescent moieties, chemiluminescent agents, radioactive isotopes and heavy metals or compounds (for example super paramagnetic iron oxide nanocrystals for detection by MRI). Methods for using detectable moieties and guidance in the choice of detectable moieties appropriate for various purposes are known to the person of ordinary skill in the art and are discussed for example in Sambrook et al. (Molecular Cloning: A Laboratory Manual, 4^{th} ed, Cold Spring Harbor, N. Y., 2012) and Ausubel et al. (In Current Protocols in Molecular Biology, John Wiley & Sons, New York, through supplement 104, 2013).

Detecting: Identification of the existence, presence, or fact of something. General methods of detecting are known to the skilled artisan and may be supplemented with the protocols and reagents disclosed herein. For example, included herein are methods of detecting a cell or tissue with increased β-oxidation of fatty acids compared to a control. Non-limiting examples of detection methods include radiolocalization, radioimaging, positron emission tomography (e.g., using an ¹⁸F-labeled fatty acid analog), magnetic resonance imaging, fluorescence imaging (e.g., using a fluorescent dye labeled fatty acid analog), and visual detection (e.g., using a chromophore-labeled fatty acid analog).

Effective amount: The amount of an agent (such as a fatty acid analog) that alone, or together with one or more additional agents, is sufficient to achieve a desired result in vitro or in vivo. For instance, this can be the amount necessary to identify a tumor in the subject, or identify a cell with altered (e.g., increased) fatty acid β-oxidation compared to a control, by detecting a detectable moiety linked to a fatty acid analog administered to the subject. Alternatively, an effective amount can be the amount of a fatty acid analog necessary to identify a cell or cells with altered (e.g., increased or decreased) fatty acid β-oxidation compared to a control in vitro, such as in tissue culture.

Several preparations disclosed herein can be administered to a subject in an effective amount. When administered to a subject, a dosage will generally be used that will achieve target tissue concentrations that has been shown to achieve a desired in vitro effect. Ideally, an effective amount provides a diagnostic effect without causing a substantial cytotoxic effect in the subject. The effective amount administered to a subject will vary depending upon a number of factors associated with that subject, for example the overall health of the subject, and the manner of administration of the therapeutic composition. An effective amount can be determined by varying the dosage and measuring the resulting response. Effective amounts also can be determined through various in vitro, in vivo or in situ assays. The disclosed compounds can be administered in a single dose, or in several doses, as needed to obtain the desired response.

Fatty Acid: a carboxylic acid having a long, unbranched, aliphatic chain or tail. Fatty acids typically contain from 4 to 22 carbon atoms (usually an even number), though more carbon atoms are also possible. Fatty acids can be represented by the general formula RCOOH, where R is a saturated or unsaturated aliphatic chain. Saturated fatty acids can be described by the general formula CH₃(CH₂)ₓCOOH.

Fatty Acid Analog: A molecule that differs in chemical structure from a parent fatty acid, but which can undergo β-oxidation in mitochondria of cells. Fatty acid analogs include homologs (differing by an increment in the chemical structure, such as a difference in the length of an alkyl chain), a molecular fragment, a structure that differs by one or more functional groups, or a change in ionization, for example. In several embodiments, the fatty acid analog is a palmitate (saturated) or oleate (monounsaturated) fatty acid analog. In several embodiments, a fatty acid analog is provided that includes a sulfur atom substituted at the C4 position, a triazole group, and detectable moiety.

Pharmaceutically Acceptable Salts or Esters: Salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

Pharmaceutical Composition: A composition that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

Subject: Any mammal, such as humans, non-human primates, pigs, sheep, cows, rodents, and the like. In two non-limiting examples, a subject is a human subject or a murine subject. Thus, the term “subject” includes both human and veterinary subjects.

Tumor, Neoplasia, or Cancer: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue or can metastasize (or both) is referred to as “malignant.”

Tumors of the same tissue type are primary tumors originating in a particular organ and may be divided into tumors of different sub-types. For example, lung carcinomas can be divided into an adenocarcinoma, small cell, squamous cell, or non-small cell tumors.

Non-limiting examples of tumors include sarcomas (connective tissue cancer) and carcinomas (epithelial cell cancer), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colorectal carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Under conditions sufficient for: A phrase that is used to describe any environment, such as an in vitro or in vivo environment, that permits a desired activity. In one example the desired activity is a click chemistry reaction.

### II. COMPOUNDS

### 1. Exemplary Labeled Fatty Acid Analogs

In some embodiments, a fatty acid analog linked to a detectable moiety can have a general formula according to structure I or II:

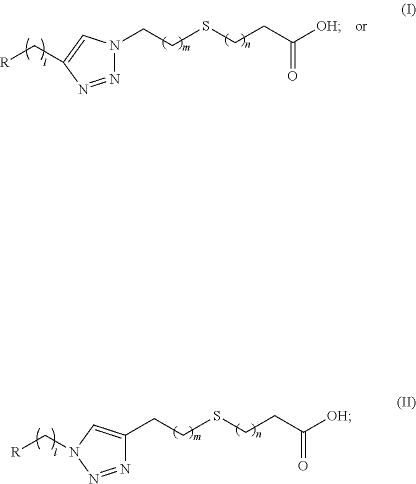

wherein R is a detectable moiety, and wherein l, m, and n independently are from 0 to 20, and wherein the carbon backbone of the compound optionally comprises one or more unsaturated double or triple bonds. In an alternative embodiment, a fatty acid analog linked to a detectable moiety can have a general formula according to structure I or II, wherein R is a detectable moiety, wherein the sum of l, m, and n is from 0 to 22 (such as from 2-20, 2-10, 2-5, 10-20, 10-15, 15-20, or 10, 11, 12, 13, 14, 15, 16, 17, 18, 19, or 20), and wherein the carbon backbone of the compound optionally comprises one or more unsaturated double or triple bonds. In some examples, l is 1-3, m is 8-14, and n is 1-3, for example l, m, and n can be 2, 5, and 1, respectively. In several embodiments, n is 2.

The incorporation of the sulfur atom in the backbone allows the intracellular trapping of the fatty acid analog, once it is oxidized through β-oxidation. The triazole functional group may form hydrogen bonding intramolecularly or intermolecularly, which increases the hydrophilicity of the molecule, allowing better pharmacokinetics. The structural changes are significant to disrupt the unwanted incorporation of the molecules into lipid bilayers

In some embodiments, the labeled fatty acid analog according to general formula (I) is labeled with a detectable moiety, such as a radioactive halogen (“*Ha”; e.g., ¹⁸F), and includes the structure of general formula (Ib):

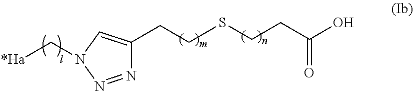

In some embodiments, the labeled fatty acid analog according to general formula (II) is labeled with a detectable moiety, such as a radioactive halogen (“*Ha”; e.g., ¹⁸F), and includes the structure of general formula (IIb):

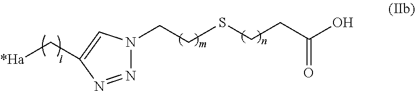

In some embodiments, the labeled fatty acid analog according to general formula (I) is labeled with a detectable moiety, such as a visible dye (e.g., a fluorophore or chromophore), such as a near-infrared (NIR) dye (“Dye”; e.g., Cy3, cypate), and includes the structure of general formula (Ic):

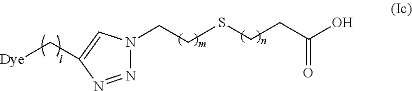

In some embodiments, the labeled fatty acid analog according to general formula (II) is labeled with a detectable moiety, such as a visible dye (e.g., a fluorophore or chromophore), such as a near-infrared (NIR) dye (“Dye”; e.g., Cy3, cypate), and includes the structure of general formula (IIc):

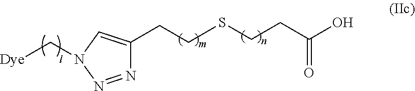

In some embodiments, the labeled fatty acid analog according to general formula (II) is labeled with detectable moiety, such as a radioactive halogen (“*Ha”; e.g., ¹⁸F), and includes the structure of formula (III):

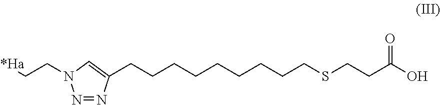

wherein Ha* is a radioactive halogen.

In some embodiments, the labeled fatty acid analog according to general formula (II) is labeled with detectable moiety, such as ¹⁸F, and includes the structure of formula (IIIa):

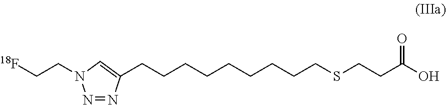

In some embodiments, the labeled fatty acid analog according to general formula (II) is labeled with a detectable moiety, such as a visible dye (e.g., a fluorophore or chromophore), such as a near-infrared (NIR) dye (“Dye”; e.g., Cy3, cypate), and includes the structure of formula (III):

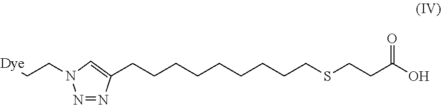

In some embodiments, the labeled fatty acid analog according to general formula (II) is labeled with a radioactive halogen (“*Ha”; e.g., ¹⁸F), and includes the structure of formula (V):

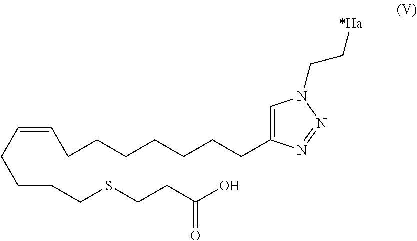

wherein Ha* is a radioactive halogen.

In some embodiments, the labeled fatty acid analog according to general formula (II) is labeled with detectable moiety, such as ¹⁸F, and includes the structure of formula (Va):

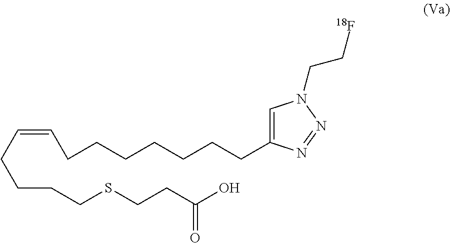

In some embodiments, the labeled fatty acid analog according to general formula (II) is labeled with a detectable moiety, such as a visible dye (e.g., a fluorophore or chromophore), such as a near-infrared (NIR) dye (“Dye”; e.g., Cy3, cypate), and includes the structure of formula (VI):

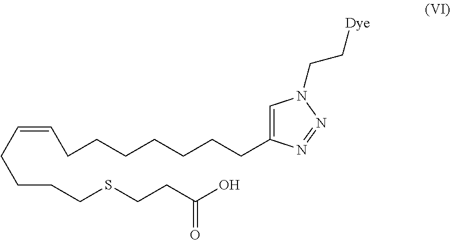

In several embodiments, the fatty acid analog is linked to detectable moieties. Suitable detectable moieties for the fatty acid analog are described and known to the skilled artisan. For example, various fluorescent materials, luminescent materials, magnetic agents, and radioactive materials can be used.

In some embodiments, the detectable moiety can be a radioactive isotope. The radiolabel may be used for both diagnostic and therapeutic purposes. Non-limiting examples of radioactive moieties that can be included as a detectable moiety on the fatty acid analog include radioactive halogens, such as ¹⁸F, ¹⁹F, ¹²³I, ¹³¹I, ¹²⁴I, ²¹¹At, ⁷⁵Br, and ⁷⁶Br.

In some embodiments, the detectable moiety can be a dye, such as a fluorescent dye that contains a fluorescent moiety. Non-limiting examples of suitable dyes include umbelliferone, fluorescein, fluorescein isothiocyanate, rhodamine, dichlorotriazinylamine fluorescein, dansyl chloride, phycoerythrin, cyan dyes such as Cy3, Cy5, Cy7, cypate, etc. In some embodiments, the fluorescent moiety emits light in the infrared, or near infrared wavelength.

The detectable moiety can be a magnetic agent, such as gadolinium. The fatty acid analog can also be labeled with lanthanides (such as europium and dysprosium), and manganese. Paramagnetic particles such as superparamagnetic iron oxide are also of use as labels.

The detectable moiety can also be a radiometal chelate, where the radiometal is ^{99m}Tc, ¹¹¹In, ⁶⁸Ga, ⁶⁴Cu, ¹⁷⁷Lu or ⁸⁹Zr.

In view of the large number of methods that have been reported for attaching a variety of radiodiagnostic compounds, radiotherapeutic compounds, and other agents to compounds, one skilled in the art will be able to determine a suitable method for attaching a given detectable moiety to the disclosed fatty acid analogs. Further, means of detecting such labels are well known to those of skill in the art. Thus, for example, radiolabels may be detected using positron emission tomography (PET), photographic film or scintillation counters; fluorescent markers may be detected using a photodetector to detect emitted illumination, and colorimetric labels are detected by simply visualizing the colored label.

### 2. Exemplary Precursors of Labeled Fatty Acid Analogs

In several embodiments, precursors of the disclosed labeled fatty acid analogs are provided, for example for use in making a disclosed fatty acid analog. A precursor is an intermediate compound or molecular complex that participates in a chemical reaction to form another compound.

In some embodiments, a precursor of the labeled fatty acid analog of general formula (I) is provided, and has the structure of general formula (VII):

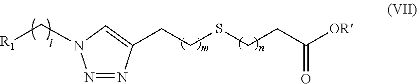

wherein R₁ is a functional group (such as a tosyl group), R′ is H or Methyl, and wherein l, m, and n independently are from 0 to 20, and wherein the carbon backbone of the compound optionally comprises one or more unsaturated double or triple bonds. In an alternative embodiment, a precursor of the labeled fatty acid analog of general formula (I) is provided, and has the structure of general formula (VII), wherein R₁ is a functional group (such as a tosyl group), R′ is H or Methyl, and wherein the sum of l, m, and n is from 0 to 22 (such as from 2-20, 2-10, 2-5, 10-20, 10-15, 15-20, or 10, 11, 12, 13, 14, 15, 16, 17, 18, 19, or 20), and wherein the carbon backbone of the compound optionally comprises one or more unsaturated double or triple bonds. In some examples the double or triple bond is present in the m portion of the backbone. In some examples, l is 1-3, m is 8-14, and n is 1-3, for example l, m, and n can be 2, 5, and 1, respectively. In several embodiments, n is 2.

In some embodiments, a precursor of the labeled fatty acid analog of general formula (II) is provided, and has the structure of general formula (VIII):

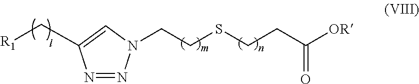

wherein R₁ is a click chemistry functional group (such as a tosyl group), R′ is H or Methyl, and wherein l, m, and n independently are from 0 to 20, and wherein the carbon backbone of the compound optionally comprises one or more unsaturated double or triple bonds. In an alternative embodiment, a precursor of the labeled fatty acid analog of general formula (II) is provided, and has the structure of general formula (VIII), wherein R₁ is a click chemistry functional group (such as a tosyl group), R′ is H or Methyl, and wherein the sum of l, m, and n is from 0 to 22 (such as from 2-20, 2-10, 2-5, 10-20, 10-15, 15-20, or 10, 11, 12, 13, 14, 15, 16, 17, 18, 19, or 20), and wherein the carbon backbone of the compound optionally comprises one or more unsaturated double or triple bonds. In some examples the double or triple bond is present in the m portion of the backbone. In some examples, l is 1-3, m is 8-14, and n is 1-3, for example l, m, and n can be 2, 5, and 1, respectively. In several embodiments, n is 2.

In some embodiments, a precursor of the labeled fatty acid analog (e.g., a precursor of the structure of formula (III)) is provided, and has the structure of formula (IX), wherein R′ is H or Methyl:

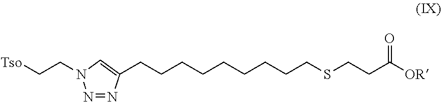

For example, the precursor can have the structure of formula (IXa) or (IXb):

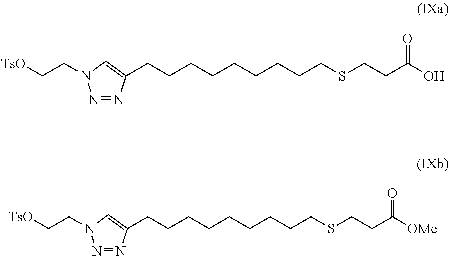 [SMILES: Cc1ccc(S(=O)(=O)OCCn2cc(CCCCCCCCCSCCC(=O)O)nn2)cc1 ; COC(=O)CCSCCCCCCCCCc1cn(CCOS(=O)(=O)c2ccc(C)cc2)nn1]

In some embodiments, a precursor of the labeled fatty acid analog (e.g., a precursor of the structure of formula (V)) is provided, and has the structure of formula (X), wherein R′ is H or Methyl:

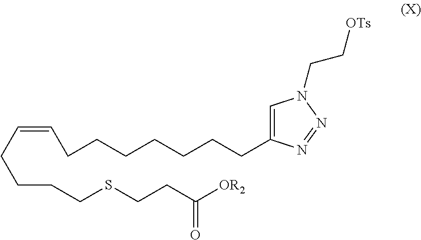

For example, the precursor can have the structure of formula (Xa) or (Xb):

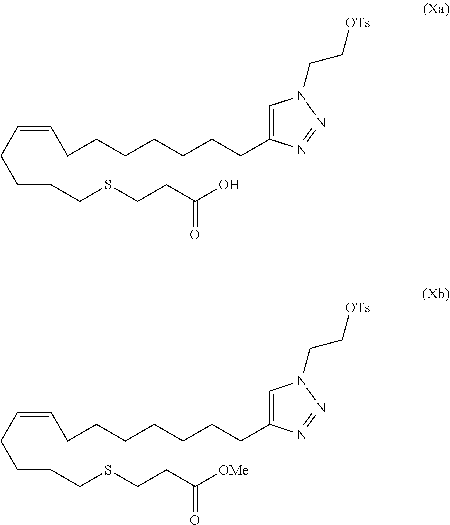 [SMILES: Cc1ccc(S(=O)(=O)OCCn2cc(CCCCCCC/C=C\CCCCSCCC(=O)O)nn2)cc1 ; COC(=O)CCSCCCC/C=C\CCCCCCCc1cn(CCOS(=O)(=O)c2ccc(C)cc2)nn1]

In some embodiments, a precursor of the labeled fatty acid analog (e.g., a precursor of the structure of formula (IV)) is provided, and has the structure of formula (XI):

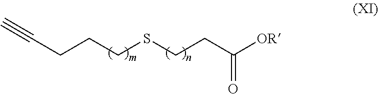

wherein R′ is H or Methyl, and m and n independently are from 0 to 20, and wherein the carbon backbone of the compound optionally comprises one or more unsaturated double or triple bonds. In an alternative embodiment, a precursor of the labeled fatty acid analog (e.g., a precursor of the structure of formula (IV)) is provided, and has the structure of formula (XI), wherein R′ is H or Methyl, and the sum of m and n is from 0 to 22 (such as from 2-20, 2-10, 2-5, 10-20, 10-15, 15-20, or 10, 11, 12, 13, 14, 15, 16, 17, 18, 19, or 20), and wherein the carbon backbone of the compound optionally comprises one or more unsaturated double or triple bonds.
In several embodiments, n is 2.

In some embodiments, the precursor of the labeled fatty acid analog (e.g., a precursor of the structure of formula (IV)) is provided, has the structure of formula (XI), wherein R′ is H or Methyl:

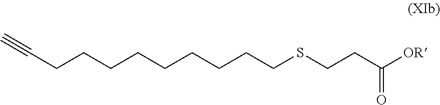

For example, the precursor can have the structure of formula (XIa) or (XIb):

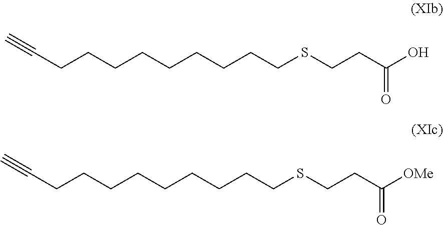 [SMILES: C#CCCCCCCCCCSCCC(=O)O ; C#CCCCCCCCCCSCCC(=O)OC]

In some embodiments, a precursor of the labeled fatty acid analog (e.g., a precursor of the structure of formula (VI)) is provided, and has the structure of general formula (XII), wherein R′ is H or Methyl:

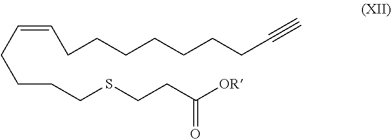

For example, the precursor can have the structure of formula (XIIa) or (XIIb):

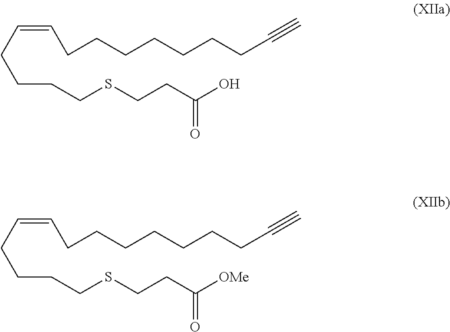 [SMILES: C#CCCCCCCC/C=C\CCCCSCCC(=O)O ; C#CCCCCCCC/C=C\CCCCSCCC(=O)OC]

Methods of making the fatty acid analogs of the disclosure are provided herein (see Example 1). Thus, in some embodiments, methods of synthesizing the fatty acid analog or precursor according to general formulas I, II, VII, or VIII, or formula III-VI, or IX-XII are provided.

Fatty acid analogs or precursors according to general formulas I, II, VII, or VIII, or formulas III-VI, or IX-XII may be in the form of salts. Such salts include salts suitable for pharmaceutical uses (“pharmaceutically-acceptable salts”), salts suitable for veterinary uses, etc. Such salts may be derived from aqueous bases (e.g., aqueous metal hydroxides or metal hydrides), as is well-known in the art. Exemplary salts described herein are sodium salts, potassium salts, magnesium salts, and calcium salts, but generically any pharmaceutically acceptable salt may be used for methods described herein.

In one embodiment, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include salts formed when an acidic proton present in the parent compound is either replaced by a metal ion (for example, an alkali metal ion, an alkaline earth metal ion or an aluminum ion) or coordinates with an organic base (for example, ethanolamine, diethanolamine, triethanolamine, N-methylglucamine, morpholine, piperidine, dimethylamine, diethylamine, triethylamine, ammonia, etc.).

Fatty acid analogs or precursors according to general formulas I, II, VII, or VIII, or formulas III-VI, or IX-XII, as well as the salts thereof, may also be in the form of solvates, for example hydrates, and N-oxides, as are well-known in the art.

### III. COMPOSITIONS AND METHODS OF USE

This disclosure includes compositions (e.g., a pharmaceutical composition) comprising at least one labeled fatty acid analog (such as a labeled fatty acid analog according to general formulas I, II, VII, or VIII, or formulas III-VI, or IX-XII). The compositions may be applied to a cell in vitro, or a pharmaceutical composition may be formulated for use in human and/or veterinary medicine and may be applied to a cell in vivo by administering an effective amount of the pharmaceutical composition to a subject. The fatty acid analog is oxidized in the cell, and remains metabolically trapped due to the sulfur atom. In cells with increased β-oxidation of fatty acids, the fatty acid analog will accumulate, and can be detected using appropriate methods (e.g., by detecting a detectable moiety linked to the analog).

### 1. Methods

The fatty acid analogs disclosed herein can be used to monitor fatty acid metabolism in a cell or tissue, for example to detect a cell or tissue with altered β-oxidation of fatty acids compared to a control in vivo or in vitro. In some embodiments, in vivo detection of a cell or tissue with increased β-oxidation of fatty acids compared to a control indicates the presence of a tumor cell or tissue in the subject, such as a prostate tumor cell or tissue.

Several epidemiological studies suggest an association between obesity and both cancer incidence and mortality (Renehan et al. The Lancet 2008, 371, 569-578; Reeves et al., BMJ 2007, 335 (7630), 1134). Not all cancers are associated with obesity and the relative risk (RR) seems to vary among cancer sites. In epidemiologic studies cancer risk is often quantified as the proportional change risk per 5 kg/m2 increase in body mass index (BMI) which is a read-out for obesity. Using this formula, the cancers most strongly associated with obesity are endometrial cancer (RR 1.52), esophageal cancer in men (RR 1.59) and renal cancer (RR 1.24 in men and RR 1.34 in women). Some studies have reported a RR for endometrial cancer as high as 6.3 in obese individuals (Calle et al., New Engl. J. Med. 2003, 348 (17), 1625-1638)). In addition, both metabolic syndrome and insulin resistance are associated with higher risk of endometrial cancer (Friedenreich et al., Cancer Epidemiology Biomarkers and Prevention 2011, 20 (11), 2384-2395).

Obesity may not only affect the risk of developing cancer but also impacts cancer survival. Overall, obesity is associated with a 52% and 88% higher mortality rate in men and women, respectively (Calle et al., New Engl. J. Med. 2003, 348 (17), 1625-1638). This increase is not solely obesity related. Patients who are obese often have co-morbidities, including stroke, cardiovascular disease, renal disease, and/or metabolic syndrome, which may contribute to higher mortality in this cohort.

In some embodiments, the methods include contacting a cell from a subject with an effective amount of one or more of the disclosed labeled fatty acid analogs. The presence (or absence) of detectable moiety from the fatty acid analog is detected in the cell. An increase of the presence of the detectable moiety in the cell compared to a control (e.g., a non-cancerous tissue) indicates that the cell or tissue has increased β-oxidation of fatty acids. Several conditions are known to be associated with increased fatty acid metabolism, including cardiovascular disease, obesity, diabetes, and cancer. In some embodiments, the method is used to detect a cell from (or in) a tumor, and detecting an increase of the presence of the detectable moiety in the cell compared to a control (e.g., a non-cancerous tissue) indicates that the cell may be a cell from a tumor.

In some embodiments, the method is used to detect cardiovascular disease in a subject (such as by detection of diseased cardiac tissue in a subject). Detecting an increase of the presence of the detectable moiety in the cardiac tissue (e.g., heart or blood vessels (e.g., arteries, capillaries, veins)) of the subject compared to a control (e.g., corresponding healthy cardiac tissue) indicates that the cardiac tissue is subject to cardiac disease.

In some embodiments, the methods include contacting a cell from a subject with an effective amount of one or more of the disclosed labeled fatty acid analogs. The presence (or absence) of detectable moiety from the fatty acid analog is detected in the cell. An increase of the presence of the detectable moiety in the cell compared to a control (e.g., a non-cancerous tissue) indicates that the cell or tissue has increased β-oxidation of fatty acids, and therefore, may be a cell from a tumor.

An effective amount is the amount of an agent (such as a labeled fatty acid analog) that alone, or together with one or more additional agents, induces a desired response, such as, labeling of cells or tissue for the purpose of detecting β-oxidation of fatty acids in the cells or tissue. In several embodiments, an effective amount of a disclosed labeled fatty acid analog or a salt thereof is administered to a subject to detect the presence (or absence) of a cell or tissue in the subject with altered (e.g., increased or decreased) β-oxidation of fatty acids compared to a control. The effective amount of a disclosed labeled fatty acid analog is administered to the subject for a sufficient amount of time for the labeled fatty acid analog to be metabolized in the subject (e.g., to undergo β-oxidation in the mitochondria in cells of the subject), and the detectable moiety included on the labeled fatty acid analog can then be detected. In some embodiments, detection of the cell or tissue with increased β-oxidation of fatty acids compared to the control detects the presence of a tumor cell or tissue in the subject, such as a prostate tumor cell or tissue.

In some embodiments, the subject has, or is suspected of having, a cell or tissue with increased β-oxidation of fatty acids (for example, a tumor, such as a prostate tumor). In some embodiments, a subject is selected who has, is suspected of having, or is at risk of developing, a tumor (such as a prostate tumor). For example, the subject has, is suspected of having, or is at risk of developing prostate cancer. The presence of a cell or tissue with increased β-oxidation of fatty acids compared to a control can be detected in these subjects using the disclosed fatty acid analogs.

In some embodiments, an effective amount of a labeled fatty acid analog is administered to a subject having a tumor before, during, and/or following anti-cancer treatment (e.g., surgery to remove the tumor). After a sufficient amount of time has elapsed to allow for the administered labeled fatty acid analog to undergo β-oxidation in the cells of the subject, the detectable moiety of the labeled fatty acid analog is detected. The labeled fatty acid analog can be administered to a subject prior to, or following, treatment of a tumor. The tumor can be (but is not limited to) a tumor from prostate cancer. An increase or decrease in the presence of the detectable moiety in the cell or tissue compared to a control can be used to determine the effectiveness of the treatment. For example, an increase or no change in the presence of the detectable moiety compared to a control taken prior to the treatment indicates that the treatment is not effective, whereas a decrease in the in the presence of the detectable moiety compared to a control taken prior to the treatment indicates that the treatment is effective. The person of ordinary skill in the art will appreciate that various controls (e.g., a positive or negative control) can be used to determine the effectiveness of an anti-cancer treatment.

In additional embodiments, a method of assaying fatty acid metabolism is provided; for example, the method can be used to identify a cell with increased or decreased β-oxidation compared to a control. The method includes contacting a cell with an effective amount of a disclosed fatty acid analog containing a terminal alkyne group (such as according to Structure XI or Alkyn-Palmitate) or salt thereof under conditions sufficient for the fatty acid analog to undergo β-oxidation in the cell; contacting the cell with an effective amount of a detectable moiety linked to an azide functional group (such as compound XIX) under conditions sufficient for 1,3-dipolar cycloaddition; and detecting the detectable moiety linked to the fatty acid analog in the cell. The cell can be in vitro or in vivo. In several in vitro embodiments, the cell can be treated with a fixative agent (such as 4% paraformaldehyde) following incubation with the fatty acid analog and prior to incubation with the detectable moiety. Detecting an increase or decrease in the detectable moiety in the cell compared to a control indicates that the cell is a cell with increased or decreased β-oxidation, respectively.

In several embodiments, the disclosed methods include detection of fatty acid β-oxidation in cells of a biological sample, for example a biopsy sample obtained from a subject having or suspected of having a tumor. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. Detecting an increase in β-oxidation of the fatty acid analog in the sample from the subject compared to a control (e.g., a control sample from a healthy subject, or a non-tumor sample) can be used to identify the subject as having increased β-oxidation (e.g., as a subject with a tumor).

### 2. Modes of Administration

The fatty acid analog can be administered to a subject in various ways, including local and systemic administration, such as, e.g., by injection subcutaneously, intravenously, intra-arterially, intraperitoneally, intramuscularly, intradermally, or intrathecally. The fatty acid analog can also be administered by direct injection at or near the site of disease.

The fatty acid analog may also be administered orally in the form of microspheres, microcapsules, liposomes (uncharged or charged (e.g., cationic)), polymeric microparticles (e.g., polyamides, polylactide, polyglycolide, poly(lactide-glycolide)), microemulsions, and the like.

A further method of administration is by osmotic pump (e.g., an Alzet pump) or mini-pump (e.g., an Alzet mini-osmotic pump), which allows for controlled, continuous and/or slow-release delivery of the fatty acid analog or pharmaceutical composition over a pre-determined period. The osmotic pump or mini-pump can be implanted subcutaneously, or near a target site.

It will be apparent to one skilled in the art that the fatty acid analog or compositions thereof can also be administered by other modes. Determination of the most effective mode of administration of the fatty acid analog or compositions thereof is within the skill of the skilled artisan. The fatty acid analog can be administered as pharmaceutical formulations suitable for, e.g., oral (including buccal and sub-lingual), rectal, nasal, topical, pulmonary, vaginal or parenteral (including intramuscular, intraarterial, intrathecal, subcutaneous and intravenous) administration, or in a form suitable for administration by inhalation or insufflation. Depending on the intended mode of administration, the pharmaceutical formulations can be in the form of solid, semi-solid or liquid dosage forms, such as tablets, suppositories, pills, capsules, powders, liquids, suspensions, emulsions, creams, ointments, lotions, and the like. The formulations can be provided in unit dosage form suitable for single administration of a precise dosage. The formulations comprise an effective amount of a therapeutic agent, and one or more pharmaceutically acceptable excipients, carriers and/or diluents, and optionally one or more other biologically active agents.

### 3. Compositions

Pharmaceutical compositions for administration to a subject can include at least one further pharmaceutically acceptable additive such as carriers, thickeners, diluents, buffers, preservatives, surface active agents and the like in addition to the fatty acid analog. Pharmaceutical compositions can also include one or more additional active ingredients such as anti-cancer agents, or an imaging agent, and the like. The pharmaceutically acceptable carriers useful for these formulations are conventional. Remington's Pharmaceutical Sciences, by E. W. Martin, Mack Publishing Co., Easton, Pa., 19th Edition (1995), describes compositions and formulations suitable for pharmaceutical delivery of the compounds disclosed herein.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical compositions disclosed herein include those formed from pharmaceutically acceptable salts and/or solvates of the disclosed fatty acid analogs. Pharmaceutically acceptable salts include those derived from pharmaceutically acceptable inorganic or organic bases and acids. In some embodiments, a fatty acid analog includes at least one basic group that can form acid-base salts with acids. Examples of basic groups include, but are not limited to, amino and imino groups. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with α-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In particular, suitable salts include those derived from alkali metals such as potassium and sodium, alkaline earth metals such as calcium and magnesium, among numerous other acids well known in the pharmaceutical art.

Certain disclosed fatty acid analogs may include at least one acidic group that can form an acid-base salt with an inorganic or organic base. Examples of salts formed from inorganic bases include salts of the presently disclosed compounds with alkali metals such as potassium and sodium, alkaline earth metals, including calcium and magnesium and the like. Similarly, salts of acidic compounds with an organic base, such as an amine (as used herein terms that refer to amines should be understood to include their conjugate acids unless the context clearly indicates that the free amine is intended) are contemplated, including salts formed with basic amino acids, aliphatic amines, heterocyclic amines, aromatic amines, pyridines, guanidines and amidines. Of the aliphatic amines, the acyclic aliphatic amines, and cyclic and acyclic di- and tri-alkyl amines are particularly suitable for use in the disclosed compounds. In addition, quaternary ammonium counterions also can be used. For additional examples of “pharmacologically acceptable salts,” see Berge et al., J. Pharm. Sci. 66:1 (1977).

To formulate the pharmaceutical compositions, the disclosed fatty acid analogs can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The disclosed fatty acid analogs can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The disclosed fatty acid analog can be combined with the base or vehicle according to a variety of methods, and release of the compound can be by diffusion, disintegration of the vehicle, or associated formation of water channels. In some circumstances, a disclosed fatty acid analog is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering a disclosed fatty acid analog can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of a disclosed fatty acid analog can be brought about by including in the composition an agent which delays absorption, for example, monostearate salts and gelatin.

In certain embodiments, a disclosed fatty acid analog can be administered in a timed release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the calcium channel agonist and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

### 4. Dosage

The dosage form of the pharmaceutical composition will be determined by the mode of administration chosen. Embodiments of the disclosed pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

Certain embodiments of the pharmaceutical compositions comprising fatty acid analogs as described herein may be formulated in unit dosage form suitable for individual administration of precise dosages. The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration. The amount of fatty acid analog administered will depend on the subject being treated, the severity of the affliction, and the manner of administration, and is known to those skilled in the art. Within these bounds, the formulation to be administered will contain a quantity of the compounds disclosed herein in an amount effective to achieve the desired effect in the subject being treated.

Embodiments of the disclosed fatty acid analogs will generally be used in an amount effective to achieve the intended result, for example an amount effective to detect increased β-oxidation of fatty acids in a cell or tissue compared to a control. In several embodiments, an amount of a disclosed fatty acid analog effective to detect a particular disease or condition, such as the presence of a tumor in a subject (e.g., a prostate tumor) is used.

The amount of compound administered will depend upon a variety of factors, including, for example, the particular indication, the mode of administration, the severity of the indication being detected, the age and weight of the patient, and the bioavailability of the particular active compound, etc. Determination of an effective dosage is well within the capabilities of those skilled in the art.

Effective dosages may be estimated initially from in vitro or in vivo assays. For example, an initial dosage may be formulated to achieve a circulating blood or serum concentration of active compound that is at or above the amount of the particular compound required to detect increased β-oxidation of fatty acids in a cell in an in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1 46, latest edition, Pagamonon Press, and the references cited therein.

Initial dosages can also be estimated from in vivo data, such as animal models Animal models useful for testing the efficacy of compounds for use in methods of detection are well-known in the art. Ordinarily skilled artisans can routinely adapt such information to determine dosages suitable for human administration.

Dosage amounts may be in the range of from about 0.0001 or 0.001 or 0.01 mg/kg bodyweight to about 100 mg/kg body weight, but may be higher or lower, depending upon, among other factors, the activity of the detectable moiety on the labeled fatty acid analog, its bioavailability, the mode of administration and various factors discussed above. A non-limiting range for an effective amount of a disclosed fatty acid analog within the methods and formulations of the disclosure is about 1.85 MBq to 370 MBq per injection dose.

Dosage can be varied by the attending clinician to maintain a desired concentration at a target site (for example, systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation. Dosage amount and interval may be adjusted individually to provide plasma levels of the compound(s) which are sufficient to provide a detectable level of the detectable moiety in the desired cell or tissue. For example, the compounds may be administered in a single bolus, or in multiple doses. In cases of local administration or selective uptake, such as local topical administration, the effective local concentration of active compound(s) may not be related to plasma concentration. Skilled artisans will be able to optimize effective local dosages without undue experimentation.

### IV. KITS

Kits are also provided. For example, a kit that contains a precursor of the radioactive compound can be labeled easily with a radiolabeled synthon for nuclear medicine imaging. The kits will typically include a disclosed fatty acid analog or salt thereof, or compositions including such molecules.

The kit can include a container and a label or package insert on or associated with the container. Suitable containers include, for example, bottles, vials, syringes, etc. The containers may be formed from a variety of materials such as glass or plastic. The disclosed fatty acid analog is optionally contained in a bulk dispensing container or unit or multi-unit dosage form. The container typically holds a composition including one or more of the disclosed fatty acid analogs or compositions. In several embodiments the container may have a sterile access port (for example the container may be an intravenous solution bag or a vial having a stopper pierceable by a hypodermic injection needle). A label or package insert indicates that the composition is used for treating the particular condition.

The label or package insert typically will further include instructions for use of the fatty acid analog or composition included in the kit. The package insert typically includes instructions customarily included in commercial packages of diagnostic products that contain information about the indications, usage, dosage, administration, contraindications and/or warnings concerning the use of such products. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as filter sets to detect fluorescent labels, or the like). The kits may additionally include buffers and other reagents routinely used for the practice of a particular method. Such kits and appropriate contents are well known to those of skill in the art.

### EXAMPLES

The following examples are provided to illustrate particular features of certain embodiments, but the scope of the claims should not be limited to those features exemplified.

### Example 1

### Fatty Acid Analogs to Image Fatty Acid Metabolism

This example illustrates novel fatty acid analogs and their use for the detection of β-oxidation of fatty acids in cells and tissue.

A number of tumors grow in the vicinity of adipocytes (e.g. breast cancer) or metastasize to the predominantly adipocyte-dominated host environment in the abdominal cavity (e.g. gastric and ovarian cancer). Studies linking adipocytes to tumorigenesis have increased in number over the last decade, most focused on breast, prostate, and colon cancer. A possible role for adipocytes in tumor development was first suggested in the mid-1960's with pioneering work by Spector (Bethesda) who showed that while 40-50% of the ¹⁴C-palmitate injected directly into the peritoneal fluid was directly incorporated into Ehrlich ascites tumor cells, only 1% of ¹⁴C-glucose was incorporated into cellular lipids. A later study examined mice harboring Ehrlich ascites tumors that were injected either IP or IV with tritiated palmitate. IP injected palmitate was rapidly incorporated into tumor cell lipids. However, if the palmitate was introduced into the plasma by IV, there was only minimal incorporation of external lipids in the tumor. The authors hypothesized that rather than using external lipids from the plasma the tumors utilized fatty acids directly transferred from the host intraperitoneal fluid (Mermier et al., J. Lipid Res., 15, 339-351, 1974).

In tumors growing in an adipose tissue-dominated microenvironment, adipocytes vanish, fibroblast-like cells accumulate, and a desmoplastic stroma ensues. As an example, in both ovarian cancer and renal cell cancer, tumor cells replace and invade the adipocytes microenvironment and the adipocytes vanish. Histological studies of these but also other abdominally metastasizing cancers (colon, gastric, pancreatic) suggest that at the invasive front of tumor cells the adipocytes become smaller and the number of fibroblast like cells increases, raising the interesting possibility that the fibroblast-like cells might be pre-adipocytes derived from dedifferentiated mature adipocytes.

Prostate cancer (PCa) is the second leading cause of cancer-related deaths in men in the United States. PCa is the most common cancer in men, accounting for one third of all cancer cases in men. It has been estimated that 1 in 3 men will be diagnosed with PCa or a precancerous prostatic lesion sometime during their lifetime. Even after treatments such as prostatectomy, external radiation, brachytherapy and cryosurgery, PCa can recur. Anatomical scans such as computerized tomography (CT) or magnetic resonance imaging (MRI) do not consistently detect early treatable or recurrent PCa, nor do they provide information on whether the cancer has the aggressive phenotype. Accurate, non-invasive, repetitive imaging of PCa will allow early detection, management of disease, and monitoring of metastasis, recurrence and therapeutic efficacy. Moreover, it will facilitate the discovery and development of therapeutic drugs towards curing PCa. Currently, the use of PET imaging in PCa is limited, mainly because of the lack of radiotracers for imaging PCa (Jadvar et al., Am. J. Roentgenol., 199 (2), 278-291, 2012). The PET tracers currently being studied for PCa imaging target glycolysis, lipogenesis, cell proliferation, and receptor proteins.

Adipocytes also play an important role in prostate cancer progression. Accordingly, the extracapsular extension of prostate cancer cells into periprostatic adipose tissue is an adverse prognostic factor. Primary murine adipocytes support colony formation in several prostate cancer cell lines, although this effect varies depending on androgen-dependence. Adipocytes may also provide lipid mediators to support prostate tumorigenesis, as evidenced by translocation of lipids from adipocytes to prostate cancer cells visualized by FTIR spectroscopy (Gazi, et al., J. Lipid Res., 48, 1846-1856, 2007). It is interesting to point out that fatty acid β-oxidation is major source of energy for prostate cancer cells (Liu et al., Prostate Cancer and Prostatic Diseases, 9, 230-234, 2006) and that glucose utilization in prostate cancer is low, limiting the utility of ¹⁸F-FDG PET imaging in well differentiated prostate cancer (Takahashi et al., Oncology, 72 (3-4), 226-233, 2007). Since it is known that PCa cells use fatty acids as their major energy source, ¹⁸F-labeled fatty acid analogs that are metabolically trapped can function as PCa PET tracers.

Thus, accurate detection is critical because it can help determine which patients will benefit from more extensive pelvic radiation. PET imaging with ¹⁸F-FDG in PCa is limited by its low sensitivity, since the glycolysis by PCa cells is slow, and the uptake of this tracer in the recurrent tumor has been shown to be similar to the uptake in postoperative scarring, or benign prostate tissue. Moreover, ¹⁸F-FDG is rapidly excreted into the urine, which interferes with PCa imaging. Other PET tracers that are used clinically for detecting PCa include ¹¹C-acetate, ¹¹C-choline, and ¹⁸F-fluorocholine whose accumulation in PCa is believed to involve their participation in cell membrane lipid synthesis. However, Kato et al. showed that neither SUV nor the early-to-late activity ratio of ¹¹C-acetate was significantly different for the normal prostate, benign prostatic hyperplasia (BPH), and prostate cancer (Kato et al., Eur. J. Nucl. Med. Mol. Imag., 29, 1492-1495, 2002). Thus, later efforts using ¹¹C-acetate- and choline-based PET were shifted to detecting relapse of prostate cancer after definitive treatment (Reske et al., Der Urologe, 45, 1240-1250, 2006; Martino et al., World J. Urol., 29, 595-605, 2011.

It was reported that ¹⁸F-fluorocholine PET/CT showed an overall sensitivity of 74% in the detection of malignant lesions with biochemical recurrence after initial therapy from a clinical study involving 250 prostate cancer patients with PSA relapse (Beheshti et al., J. Nucl. Med., 54, 833-840, 2013). Imaging prostate specific membrane antigen (PSMA) has shown considerable promise. The monoclonal antibody against the extracellular domain of PSMA J591 was labeled with ⁸⁹Zr-desferrioxamine B (DFO)-7E11 and evaluated for immunoPET imaging of PCa (Holland et al., J. Nucl. Med., 51, 1293-1300, 2010). ¹⁸F-labeled small molecules such as [¹⁸F]DCFBC (Mease et al., Clin. Cancer. Res., 14, 3036-3043, 2008) and ¹⁸F-fluorobenzamido-phosphoramidate (Lapi et al., J. Nucl. Med., 50, 2042-2048, 2009) were also evaluated for imaging PSMA of PCa. A gap remains in diagnosing and managing PCa through imaging its up-regulated fatty acid metabolism (Nieman et al., BBA—Molecular and Cell Biology of Lipids, 1831, 1533-1541, 2013; Santos et al., FEBS J., 279, 2610-2623, 2012). In PCa, the increased expression of fatty acid synthase (FAS; Baron et al., J. Cell. Biochem., 91, 47-53, 2004) and decreased expression of stearoyl-CoA desaturase (SCD; Moore et al., Int. J. Cancer, 114, 563-571, 2005) resulted in increased levels of palmitate which drives fatty acid oxidation. Fatty acids undergo β-oxidation in peroxisomes or mitochondria depending on their length. Fatty acids with more than 22 carbons are believed to be oxidized first in the peroxisomes by enzymes such as DBP and ACOX3, both of which are overexpressed in PCa (Zha et al., The Prostate, 63, 316-323, 2005). The consistent and specific overexpression of AMACR in PCa over normal prostate also indicates enhanced β-oxidation pathway of fatty acids (Luo et al., Cancer Res., 62, 2220-2226, 2002).

In 1967, Spector showed that 40-50% of ¹⁴C-labeled palmitate was incorporated into Ehrlich ascites tumor cells with only 1% incorporation of ¹⁴C-glucose (Spector, Cancer Res., 27, 1580-1586, 1967). In 1974, an in vivo study using the same tumor cell line showed that intraperitoneally injected palmitate was rapidly incorporated into tumor cells, though there was only minimal incorporation of when injected intravenously. The authors hypothesized that the tumor cells utilized fatty acids directly from the host intraperitoneal fluid instead of from the plasma (Mermier et al., J. Lipid Res., 15, 339-351, 1974). This was one of the first studies demonstrating the promise of labeled fatty acid to detect cancers. In 1968, Dr. Josef Pacak, et al. described for the first time, the synthesis of FDG. In the 1970s, Tatsuo Ido and Al Wolf were the first to describe the synthesis of FDG labeled with ¹⁸F. In 1976, ¹⁸F-FDG was first administered to two normal human volunteers by Abass Alavi at the University of Pennsylvania. From then on, the detection of tumor using ¹⁸F-FDG flourished. Possibly because of the success of ¹⁸F-FDG in detecting oncologic sites, fewer people were investigating alternative metabolic pathways that provide energy to tumor cells (Cantor et al., Cancer Discovery, 2, 881-898, 2012), such as glutamine (Qu et al., J. Am. Chem. Soc., 133, 1122-33, 2011) and fatty acids (Nieman et al., BBA—Molecular and Cell Biology of Lipids, 1831, 1533-1541, 2013).

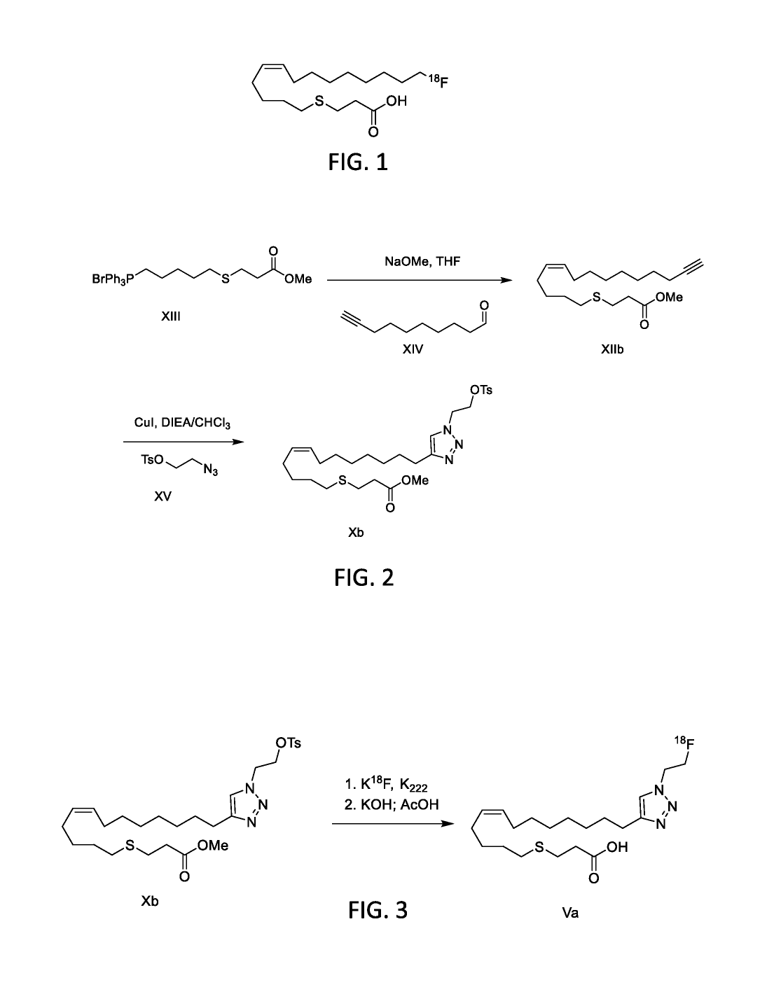

In 2006, Liu et al. showed that fatty acid oxidation is a dominant bioenergetics pathway in prostate cancer (Liu, Prostate Cancer Prostatic Dis, 9, 230-234, 2006). In 2010, Liu et al. compared the uptake of ³H-labeled fluoro-2-deoxyglucose, ¹⁸F-FDG and ³H-palmitic acid in PCa cells, which showed dominant uptake of ³H-palmitic acid rather than 2-DG (Liu et al., Anticancer Res., 30, 369-374, 2010). At about the same time, Pandey et al. released their preliminary study of 16-[¹⁸F]fluoro-4-thia-palmitic acid (FTP) uptake in three cancerous cell lines: 9L rat glioma, LNCaP, and PC3 under normoxic or hypoxic conditions (Pandey et al., Heart and Metabolism, 51, 15, 2011). The advantage of using FTP and other related fatty acid analogs is that they undergo β-oxidation and are trapped intracellularly. The trapping mechanism is very desirable in PET imaging, because the non-trapped fatty acids will release the ¹⁸F-fluoride after being completely oxidized, diminishing the signal to background ratio. However, studies using the trappable fatty acid analogs were directed to myocardial imaging rather than oncological imaging (Pandey et al., J. Med. Chem., 55, 10674-10684, 2012). [¹⁸F]Fluoro-4-thia-oleic acid (FTO) is a known PET tracer that undergoes β-oxidation in the mitochondria, is trapped intracellularly, and is used for FTO is used for cardiac imaging (see FIG. 1; Pandey et al., J. Med. Chem., 55, 10674-10684, 2012; DeGrado et al., J. Nucl. Med., 51, 1310-1317, 2010).

Optical imaging of prostate cancer provides an alternative to PET imaging. It has better resolution, allowing studies at cellular level. Near-IR fluorescent probes can be used as in vivo imaging agents without giving patients radioactive materials. Endoscope can be used to allow detection inside of the body.

Adipocytes and Fatty Acids in Colon, Ovarian, and Other Cancer Types.

The biology of ovarian cancer is different from breast, colon, and prostate cancer, since distant metastasis is rare and the cancer is confined to the peritoneal cavity (Lengyel et al., Am. J. Pathol., 177, 1053-1064, 2010). The most common site of metastasis is the human omentum, a large fat pad, reaching the pelvis and positioned in front of the small bowel, that is 20×20×10 cm in size. In an effort to understand why ovarian cancer metastasizes to the omentum, Lengyel and co-workers showed that primary human omental adipocytes induce breast, colon, and ovarian cancer cell proliferation and invasion in vitro and ovarian cancer growth in vivo (Nieman et al., Nat. Med., 17, 1498-1503, 2011). In studies which attempted to explain why ovarian cancer preferentially metastasizes to the omentum, it was found that primary human omental adipocytes induce breast, colon, and ovarian cancer cell proliferation and invasion in vitro and ovarian cancer growth in vivo (Nieman et al., Nat. Med., 17, 1498-1503, 2011). It became apparent that adipocyte-secreted cytokines (IL-8 and IL-6) attract ovarian cancer cells to the omentum when the cytokine receptors responsible for binding IL-6 and IL-8 were blocked and fewer cancer cells homed to the omentum. These data showed that the cytokines secreted by the adipose tissue attract cancer cells. Once the ovarian cancer cells interact with the adipocytes they initiate HSL-mediated lipolysis in the adipocyte, releasing fatty acids, which are then taken up by the ovarian cancer cells for energy production. This was observed as an increase in fatty acid β-oxidation which was inhibited by the CPT1 inhibitor, etomoxir. These data are direct evidence for high-energy lipids provided by the adipocytes stimulating mitochondrial metabolism in the cancer cells, supporting fast tumor growth. Lipid accumulation was also evident in breast and colon cancer cells cultured with adipocytes and metastatic human ovarian cancer tissue sections (Nieman et al., Nat. Med., 17, 1498-1503, 2011). Furthermore, there is evidence that an abundant adipocyte protein, fatty acid binding protein 4 (FABP4, aP2, or A-FABP), may support tumor growth. Metastatic tumor burden was drastically reduced in a FABP4-deficient mouse model of ovarian cancer as compared to wild-type controls (Nieman et al., Nat. Med., 17, 1498-1503, 2011).

Labeled Oleate Analogs.

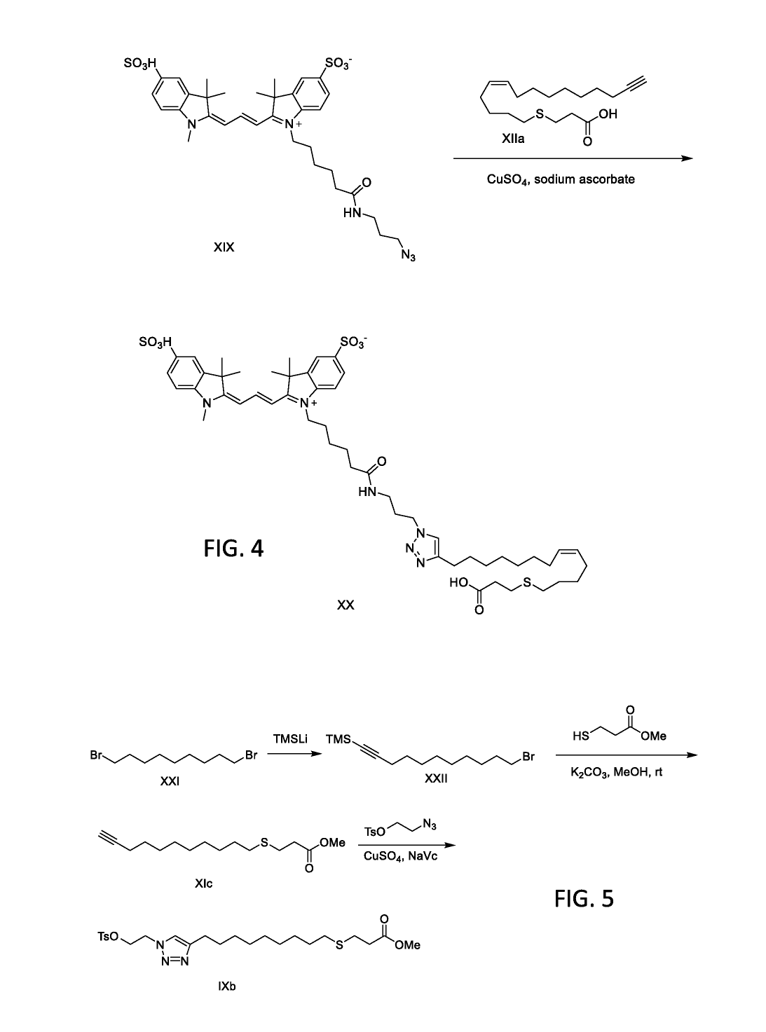

Novel oleate analogs including a triazole moiety (and precursors for making these analogs) were generated to improve pharmacokinetics and radiolabeling conditions compared to ETO (see FIGS. 2-4). A scheme for generating analogs of oleic acid (and precursors for making these analogs) that include a detectable moiety, a triazole moiety and a sulfur atom is provided in FIGS. 2-4. The analogs were designed for labeling with a radioactive halogen atom (e.g., ¹⁸F as shown in FIG. 3), or a detectable dye (e.g., Cy3 shown in FIG. 4). These analogs are useful, for example, as PET tracers to identify cells with increased β-oxidation of fatty acids (e.g., tumor cells). A brief discussion of non-limiting methods for making the disclosed oleate analogs follows:

(Z)-Methyl 3-(pentadec-5-en-14-yn-1-ylthio)propanoate (XII): To a solution of methyl 3-((5-(bromotriphenylphosphoranyl)pentyl)thio)propanoate (1.8 g, 3.39 mmol) in 6.8 ml at room temperature (rt), anhydrous THF was added NaOMe (0.37 g, 6.78 mmol). The solution turned yellow with a white suspension. After stirring vigorously for 5 min at room temperature, dec-9-ynal (0.52 g, 3.39 mmol) was added drop wise. The reaction mixture turned colorless with a white suspension, stirred for 9 hours at room temperature and was then quenched with an aqueous solution of NH₄Cl. The reaction mixture was extracted with Et₂O (20 ml×3), washed with brine (20 ml), dried with anhydrous MgSO₄, filtered, and concentrated on a rotary evaporator (rotavap), resulting in a pale yellow oil as the product (0.46 g, 42%).

(Z)-Methyl 3-((13-(1-(2-(tosyloxy)ethyl)-1H-1,2,3-triazol-4-yl)tridec-5-en-1-yl)thio)propanoate (X): To a solution of (Z)-methyl 3-(pentadec-5-en-14-yn-1-ylthio)propanoate (142 mg, 0.438 mmol) and 2-azidoethyl 4-methylbenzenesulfonate (106 mg, 0.438 mmol) in CHCl₃ (1 ml) at room temperature, CuI (8 mg, 0.044 mmol) was added followed by N,N-Diisopropylethylamine (282 mg, 2.19 mmol). The reaction mixture was filtered through a short silica plug after stirring overnight, rinsed with DCM (100 ml) and concentrated on a rotavap. The resulting mixture was dissolved in acetonitrile (ACN) and purified with a pre-packed C18 column (30 g) using a gradient of 10% ACN/H₂O (0.1% TFA) to 90% ACN/H₂O (0.1% TFA) over 15 min at a flow rate of 35 ml/min. The eluent was monitored by UV at 230 nm and 254 nm. The fraction at 15 min was collected and concentrated on a rotavap to get the pure product (178 mg, 72%). ESI-MS: 566.80 [M+H].

(Z)-3-((13-(1-(2-[¹⁸F]fluoroethyl)-1H-1,2,3-triazol-4-yl)tridec-5-en-1-yl)thio)propanoic acid (Va): Cyclotron-produced [¹⁸F]fluoride (˜600 mCi) was dried under nitrogen at 110° C. in a 3 ml glass vial containing Kryptofix 2.2.2 (10 mg), ACN (0.8 ml), and an aqueous solution of K₂CO₃ (4 mg) in water (0.2 ml). Then it was azeotropically dried with ACN (2×1 ml) at 110° C. under a continuous flow of nitrogen gas. ACN (1 ml) was added to the final [¹⁸F]fluoride residue, and then it was added to a solution of labeling precursor (2 to 4 mg) in 250 μl ACN. The reaction vessel was sealed and heated at 75° C. for 10 min After cooling on ice for 1 min, the incorporation of ¹⁸F into the methyl ester intermediate was determined by radio-TLC with ethyl acetate as the eluting solvent. The hydrolysis of the ester intermediate was carried out with aqueous KOH solution (0.15 ml, 0.2 N) at 75° C. for 5 min, cooled on ice for 1 min and then acidified with 0.15 ml acetic acid. The crude mixture was purified by semi-preparative HPLC to get the labeled product. The fraction containing the product was diluted with 50 ml water and passed through a C18 SepPak cartridge, washed with 10 ml water, and eluted with 1 ml ethanol. The volume was evaporated at 110° C. under a continuous stream of nitrogen gas to ˜200 μl and diluted with 1% BSA (lipid free) in saline for cell or animal studies.

Compound XX: A solution of (Z)-3-(pentadec-5-en-14-yn-1-ylthio)propanoic acid (1.74 μmol) and sulfo-Cy3 azide (0.174 μmol) in 100 μl t-BuOH/H₂O (1:1) was added to a suspension of CuTBTA ((0.174 μmol) and sodium ascorbate (0.87 μmol) in 50 μl t-BuOH/H₂O (1:1) at room temperature. After votexing for 10 min, the extent of reaction was determined by HPLC. Acetic acid (50 μl) was added to acidify the mixture, and then it was purified by analytical HPLC using a gradient of 20% to 90% ACN/H₂O, with 0.1% TFA; flow rate: 1.5 ml/min; product retention time: 9 min. The product was obtained as a red oil (49 nmol, 28%). ESI-MS: 1008.92 [M+H].

Labeled Palmitate Analogs.

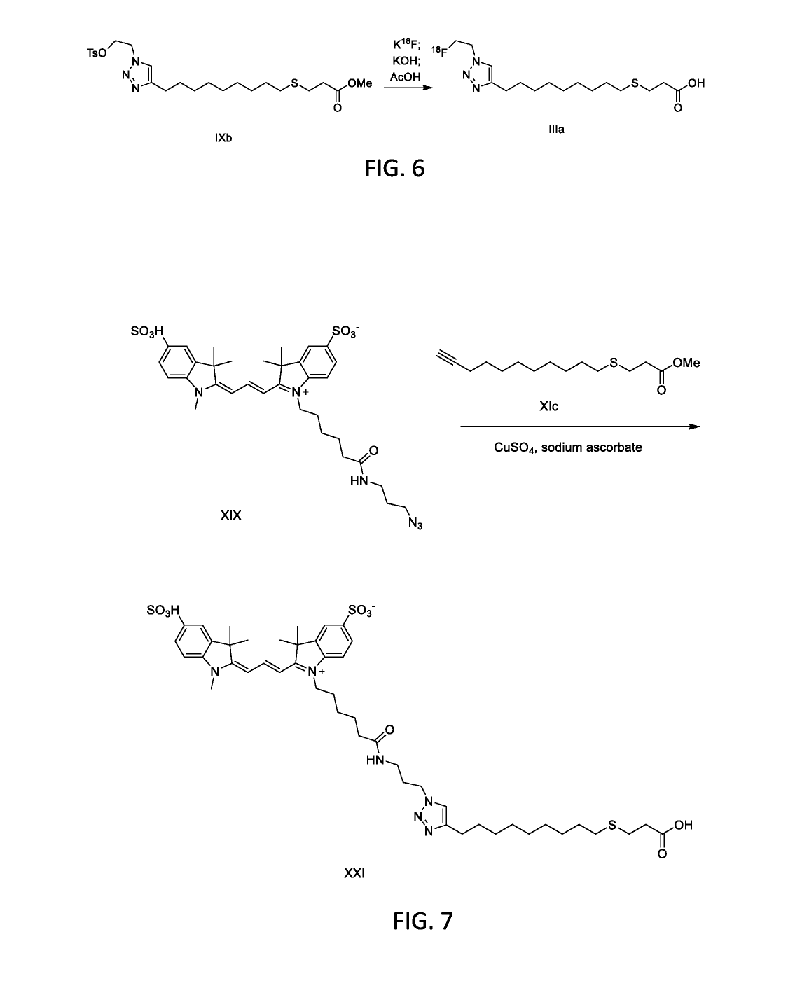

Further, analogs of FTO on a palmitate background (and precursors for making these analogs) including a detectable moiety, a triazole moiety and a sulfur atom is provided in FIGS. 5-7. The analogs were designed for labeling with a radioactive halogen atom (e.g., ¹⁸F as shown in FIG. 6), or a detectable dye (e.g., Cy3 shown in FIG. 7, compound XXI). A scheme for generating these oleic acid analogs is provided in FIGS. 5-7). These analogs are useful, for example, as PET tracers to identify cells with increased β-oxidation of fatty acids (e.g., tumor cells). A brief discussion of non-limiting methods for making the disclosed palmitate analogs follows:

(11-Bromoundec-1-yn-1-yl)trimethylsilane (XXII) was synthesized by the following literature procedure.

Methyl 3-(undec-10-yn-1-ylthio)propanoate (IX): To a solution of methyl 3-mercaptopropanoate (1.23 g, 10.2 mmol) and K₂CO₃ (2.35 g, 17 mmol) in 17 ml MeOH at room temperature, (11-bromoundec-1-yn-1-yl)trimethylsilane (0.45 g, 1.48 mmol) was added, stirred for 1 day, filtered through a short Celite plug and then concentrated on a rotavap. The crude mixture was purified by flash chromatography using 10% ethyl acetate/hexane as the eluting solvent. The pure product was obtained as a colorless oil (0.238 g, 57%). ESI-MS: 325.01 [M+H].

Methyl 3-((9-(1-(2-(tosyloxy)ethyl)-1H-1,2,3-triazol-4-yl)nonyl)thio)propanoate (XI): To a solution of methyl 3-(undec-10-yn-1-ylthio)propanoate (40 mg, 0.148 mmol), 2-azidoethyl 4-methylbenzenesulfonate (36 mg, 0.148 mmol) in CHCl₃ (1 ml) at room temperature, CuI (3 mg, 0.044 mmol) was added followed by 1 ml N,N-diisopropylethylamine. The crude reaction mixture was filtered through a short Celite plug and purified with silica gel using 1% TEA/ethyl acetate to obtain the product as an oil (66 mg, 87%). ESI-MS: 511.79 [M+H].

3-((9-(1-(2-[¹⁸F]fluoroethyl)-1H-1,2,3-triazol-4-yl)nonyl)thio)propanoic acid (XXIVa): The ¹⁸F-labeling procedure is the same as described above for the oleate analog. Briefly, cyclotron-produced [¹⁸F]fluoride (˜600 mCi) was dried down under nitrogen at 110° C. in a 3 ml glass vial containing Kryptofix 2.2.2 (10 mg), ACN (0.8 ml), and an aqueous solution of K₂CO₃ (4 mg; 0.2 ml). The resulting mixture was azeotropically dried with ACN (2×1 ml) at 110° C. under continuous flow of nitrogen gas. ACN (1 ml) was added to the final [¹⁸F]fluoride residue, and it was added to a solution of labeling precursor (2 to 4 mg) in 250 μl ACN and was sealed and heated at 75° C. for 10 min After cooling on ice for 1 min, the incorporation of ¹⁸F into the methyl ester intermediate was determined by radio-TLC with ethyl acetate as the eluting solvent. The hydrolysis of the ester intermediate was carried out with aqueous KOH solution (0.15 ml, 0.2 N) at 75° C. for 5 min, cooled on ice for 1 min and then acidified with 0.15 ml acetic acid. The crude mixture was purified by semi-preparative HPLC to get the labeled product. The fraction containing the product was diluted with 50 ml water and passed through a C18 SepPak cartridge, washed with 10 ml water, and eluted with 1 ml ethanol. The volume was evaporated at 110° C. under a continuous stream of nitrogen gas to ˜200 μl and diluted with 1% BSA (lipid free) in saline for cell or animal studies.

Sulfo-Cy3-Labeled Metabolically Trappable Fatty Acid Analog.

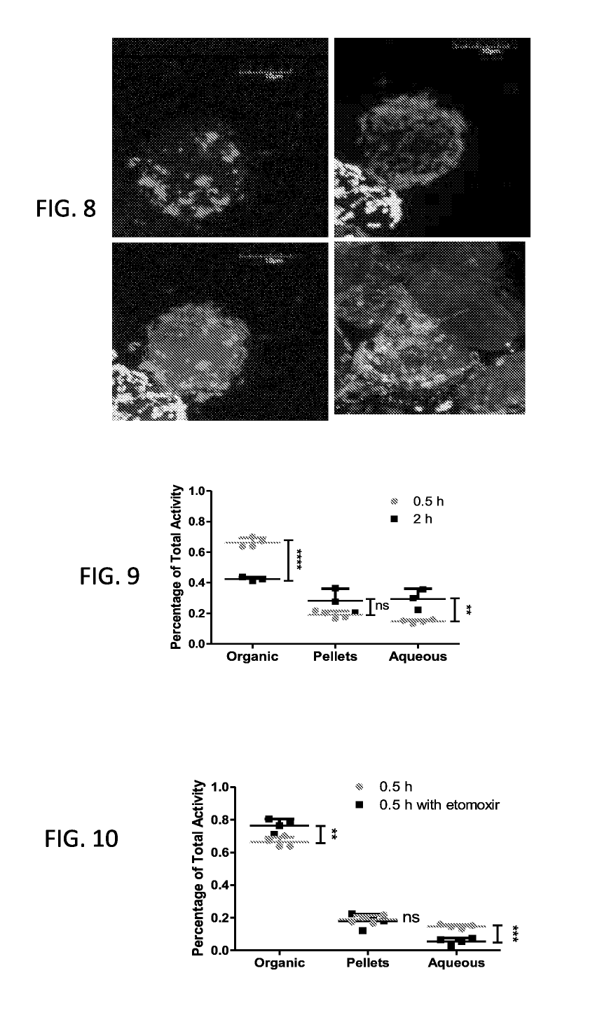

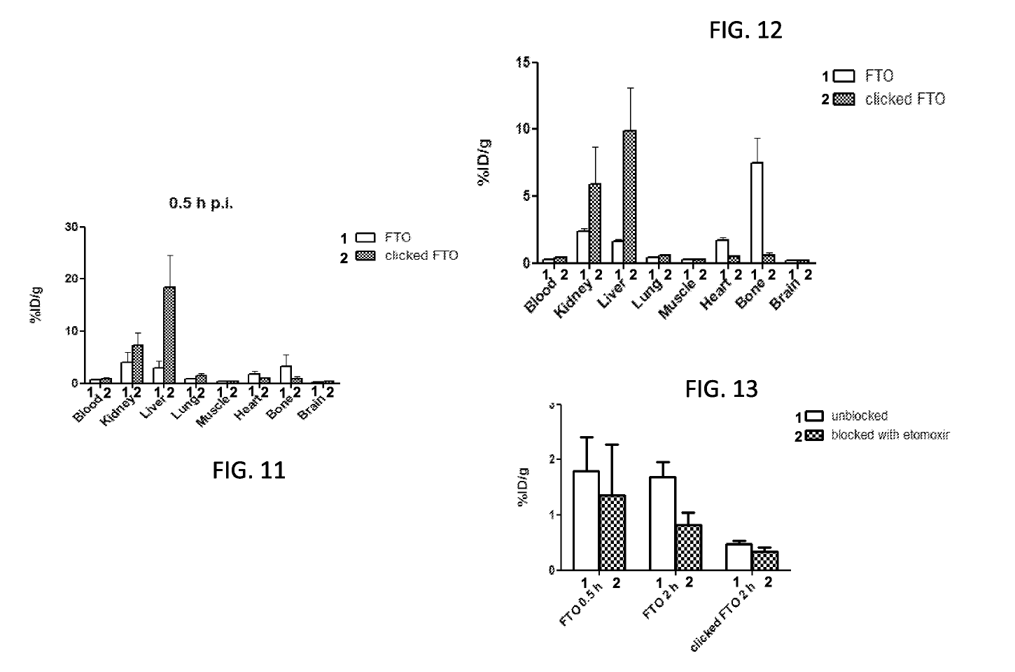

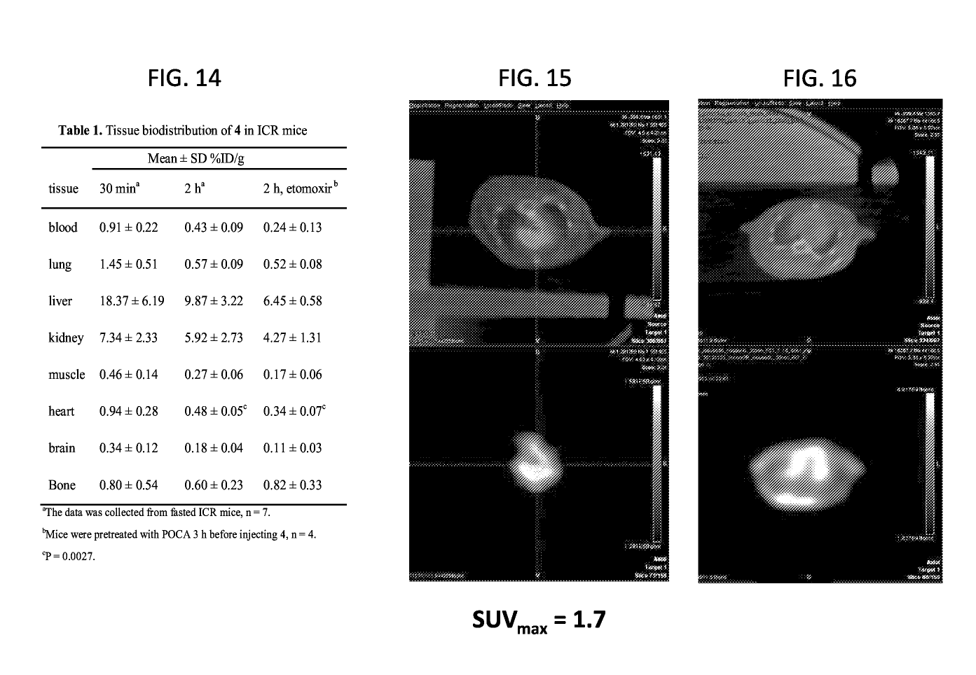

The Cy3-labeled oleate analog (compound XX; FIG. 4) was synthesized, and the cell uptake of this compound was evaluated in different tumor cell lines, including PC3 cells (a prostate cancer cell line) (FIG. 8). The PC3 cells' mitochondria were transfected with green fluorescent protein. The yellow spots indicate the co-localization of the red fatty acid with the green mitochondria where the oxidation of fatty acid happens (FIG. 8). This means the fluorescent fatty acid was taken up into the PC3 cells, and oxidized in the mitochondria, indicating that prostate cancer cells uses fatty acid as a predominant energy source, and that fatty acid analogs can be used as an imaging agent to identify for prostate cancer cells.

### Example 2

### In Vivo Analysis of Fatty Acid Metabolism Using ¹⁸F-Clicked Palmitate Analog and ¹⁸F-Clicked Oleate Analog

This example illustrates in vivo analysis of fatty acid metabolism in mice using the ¹⁸F-clicked palmitate and ¹⁸F-clicked oleate analogs.

Small animal PET/CT imaging was performed as previously described (Sprague et al., Clin. Cancer. Res. 2004, 10 (24), 8674-8682). Briefly, the ¹⁸F-clicked palmitate or ¹⁸F-clicked oleate analog (3.7 MBq) was injected via tail vein and the mouse was imaged at 2 hours postinjection. Static imaging was performed on an Inveon PET/CT scanner (Constantinescu et al., Phys. Med. Biol., 54, 2885-2899, 2009; Kemp et al., Phys. Med. Biol., 54, 2359-2376, 2009) with 10 min PET scanning followed by a 5 min CT. Inveon Research Workplace (IRW) from Siemens Healthcare Global was used for co-registration of PET/CT images and quantification of regions of interest (ROI). PET/CT images were reconstructed with maximum a posteriori (MAP), 3D ordered-subset expectation maximization (OSEM3D), 2D ordered-subset expectation maximization (OSEM2D), and filtered back projection (2DFBP). Standard uptake values (SUV) were generated by measuring ROI from PET/CT images and calculated with the formula: SUV=[nCi/ml]×[animal weight (g)]/injected dose [nCi]. FDG was injected intraperitoneally 36 hours pre-injection. 2-DG was injected intraperitoneally 4 hours pre-injection.

Myocardial cells utilize fatty acid as the main energy source under fasting conditions. FIGS. 15 and 16 are transaxial PET images of heart tissue of fast ICR mice treated with ¹⁸F-clicked palmitate analog (Compound IIIa) and 18F-clicked oleate analog (Compound Va). The bottom images are from the control group. The heart uptake of the ¹⁸F-clicked-FTP demonstrates the utility of imaging β-oxidation of fatty acids in the myocardium in disease states such as ischemia.

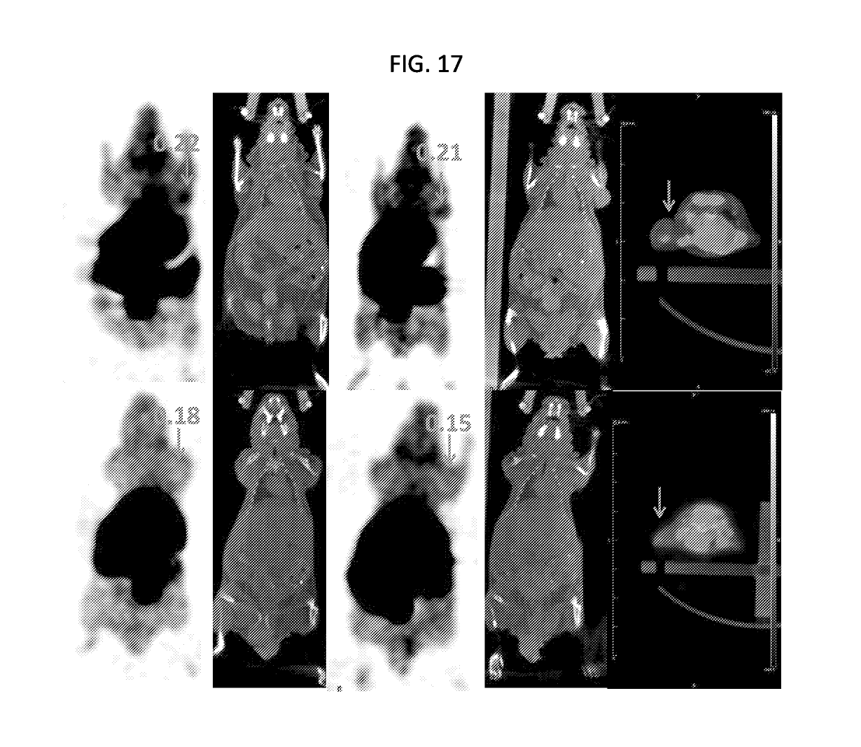

Since it is known that prostate cancer cells use fatty acids as their major energy source (Liu et al., Anticancer Res., 30, 369-374, 2010), we tested with ¹⁸F-clicked palmitate analog (Compound IIIa) that are partially metabolically trapped in a prostate cancer (PCa) xenograft model. Based on prior data of ¹⁸F-FDG and ¹¹C-acetate (Vāvere et al., J. Nucl. Med., 49, 327-334, 2008), low uptake of the fatty acid analog in subcutaneous PCa xenograft tumors was expected, as tumor cells seem to change their metabolism profile as subcutaneous xenografts. Unlabeled FDG and 2-DG were pre-injected intraperitoneally to inhibit the glycolysis of subcutaneous tumors, thereby increasing the uptake and oxidation of the labeled fatty acid analog (Sabra et al., Cancer Res., 70, 2465-2475, 2010). The 2-DG and FDG treated mice had higher uptake of ¹⁸F-clicked-FTO in the tumor (SUV=0.21, 0.22) than the control group (SUV=0.15, 0.18; P=0.0436), which was not treated with 2-DG and FDG (FIG. 17). These data are consistent with the understanding that the inhibition of glycolysis of subcutaneous tumors would increase the uptake and oxidation of the ¹⁸F-clicked palmitate analog as the alternative energy for tumor cells.

### Example 3

### In Vitro Detection of β-Oxidation

This example illustrates in vitro analysis of fatty acid metabolism using the disclosed compounds. Cells were incubated with a fatty acid analog including a terminal alkyne group, fixed, and then treated with sulfo-Cy3-azide. The sulfo-Cy3-azide undergoes a click chemistry reaction (a 1,3-dipolar cycloaddition) with the terminal azide of the fatty acid homolog in the fixed cells to form a triazole ring linked to the fatty acid analog. The Cy3 moiety is then detected by fluorescence microscopy.

### EXEMPLARY METHOD

1. Place approximately 500 PC3 cells on coverslip in 24 well plates.
2. Starve the cell with 250 μL of glucose free media overnight.
3. Replace the media before the experiment with fresh 250 μL of glucose free media.
4. Add alkyne fatty acid to the selected wells with coverslip and 1% BSA 10 μL in the control well and incubate at 37° C. for 1 or 2 h.
5. After respective time wash the cells with 500 μL of 1% BSA for 3×5 min to remove extra fatty acid.
6. Fix the cells with 300 μL of 4% paraformaldehyde (PFA) for 15 min
7. Wash the cells once with PBS.
8. Add 300 μL of 25 mM of ammonium chloride for 5 min to remove extra PFA and open the site for click chemistry.
9. Permeabilize with 300 μL of 0.5% Triton X 100 in PBS for 15 min
10. Wash once with PBS and add 250 μL of PBS.
11. Click with 12.5 μL of 1 mM Cy3 azide dye and 33 μL of 1 M sodium ascorbate and 32.7 mM copper catalyst (HB₄OH:H₂O:DMF 1:1:1) solution mixture for 1 or 2 hours on shaker.
12. Wash the well with 500 μL of 0.05% of Tween 20 in PBS for 3×5 min or until all the dye washed out.
13. Add DAPI (4 μL to 1 mL dilution), 250 μL to each well for 15 to 30 min
14. Wash DAPI with PBS for 3×5 min
15. Mount the coverslip and analyze by fluorescence microscopy.

### DISCUSSION

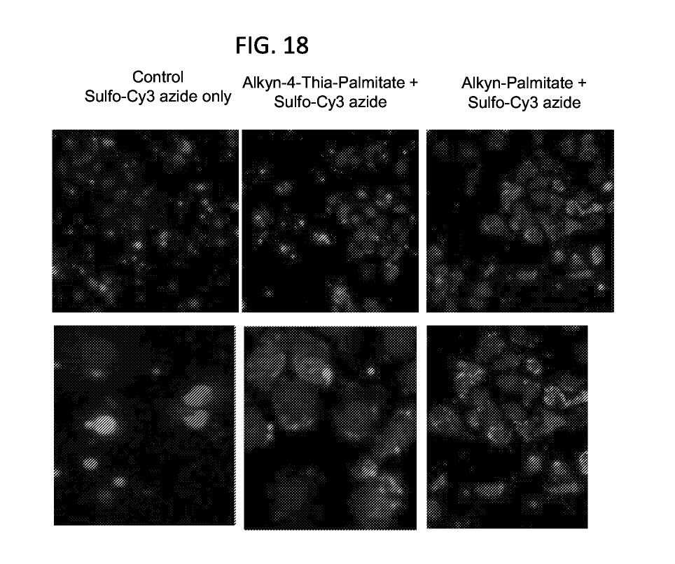

The ω-alkyne intermediate alkyn-4-thia-palmitate (Compound XIa) was designed for fluorescent microscopic imaging of fatty acids by adapting a post-fix click strategy. Alkyn-4-thia-palmitate was incubated with PCa cells. Extracellular alkyn-4-thia-palmitate was washed away. Then cells were fixed, permeated, and stained with sulfo-Cy3-azide (Compound XIX). The fluorescent microscopic imaging (FIG. 18) showed that the PCa cells avidly take up the fatty acid analog alkyn-4-thia-palmitate, suggesting that fatty acids are the energy source for those cells. The utility of alkyn-4-thia-palmitate for imaging fatty acid uptake/oxidation in cells or tissues would be applicable to other types of tumor cells besides PCa cells.

It will be apparent that the precise details of the methods or compositions described may be varied or modified without departing from the spirit of the described embodiments. We claim all such modifications and variations that fall within the scope and spirit of the claims below.

1. A fatty acid analog or salt thereof comprising a structure according to general formula I or II: 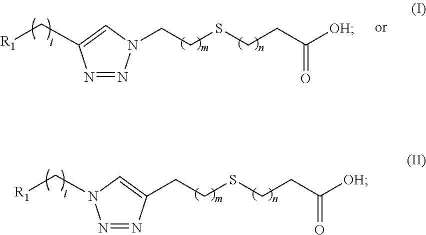
wherein R is a detectable moiety, and wherein l, m, and n independently are from 0 to 20, and wherein the carbon backbone of the compound optionally comprises one or more unsaturated double or triple bonds.

2. The fatty acid analog or salt thereof of claim 1, wherein R is radiolabeled halogen atom.

3. The fatty acid analog or salt thereof of claim 2, wherein R is ¹⁸F.

4. The fatty acid analog or salt thereof of claim 1, wherein R is a fluorescent moiety.

5. The fatty acid analog or salt thereof of claim 4, wherein R is Cy3.

6. The fatty acid analog or salt thereof of claim 1, wherein n is 2.

7. The fatty acid analog or salt thereof of claim 1, wherein the carbon backbone of the compound comprises one unsaturated double bond.

8. The fatty acid analog or salt thereof of claim 1, wherein the fatty acid analog is a palmitate or oleate analog.

9. The fatty acid analog or salt thereof of claim 1, comprising a structure according to one of formulas (III)-(VI), or a pharmaceutically acceptable salt thereof: 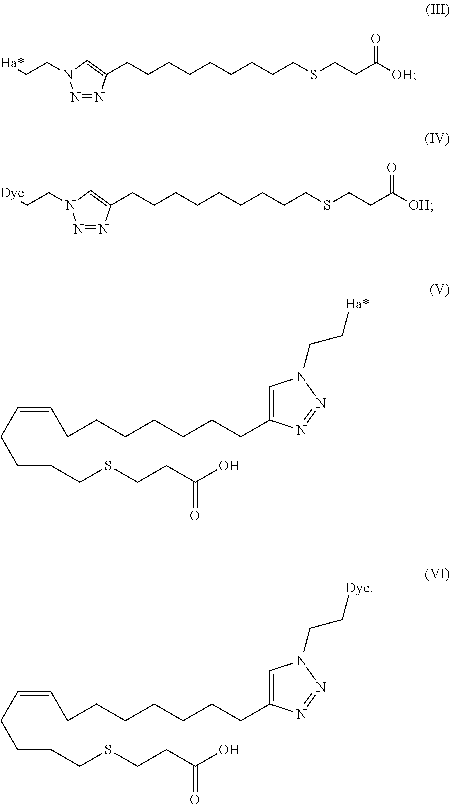
wherein HA* is a radioactive halogen and Dye is a fluorescent or visible marker.

10. The fatty acid analog or salt thereof of claim 9, wherein Ha* is ¹⁸F

11. The fatty acid analog or salt thereof of claim 9, wherein Dye is Cy3.

12. A composition, comprising:
an effective amount of the compound or salt thereof of claim 1; and
a pharmaceutically acceptable carrier.

13. A method, comprising:
contacting a cell with an effective amount of the composition of claim 12, and detecting the detectable moiety on the fatty acid analog in the cell.

14. A method, comprising:
(a) contacting a cell with an effective amount of a fatty acid analog or salt thereof comprising a structure according to general formula XI: 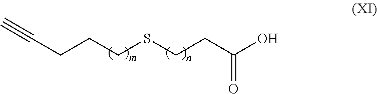
wherein m and n independently are from 0 to 20, and wherein the carbon backbone of the compound optionally comprises one or more unsaturated double or triple bonds.
(b) contacting the cell with an effective amount of a detectable moiety linked to an azide functional group under conditions sufficient for 1,3-dipolar cycloaddition; and
(c) detecting the detectable moiety linked to the fatty acid analog in the cell.

15. The method of claim 14, wherein the detectable moiety is a radiolabeled halogen atom or a fluorescent moiety.

16. The method of claim 14, wherein n is 2.

17. The method of claim 14, wherein the carbon backbone of the compound comprises one unsaturated double bond.

18. The method of claim 14, wherein the fatty acid analog is a palmitate or oleate analog.

19. The method of claim 13, wherein the cell is in vivo.

20. The method of claim 13, wherein the cell is in vitro.

21. The method of claim 14, wherein the cell is in vivo.

22. The method of claim 14, wherein the cell is in vitro.

23. A method of detecting a cell or tissue in a subject with increased β-oxidation of fatty acids, comprising:
administering an effective amount of the fatty acid analog or salt thereof of claim 1 to the subject; and
detecting an increase in the presence the detectable moiety in the cell or tissue of the subject compared to a control;
thereby detecting the cell or tissue in the subject with increased β-oxidation of fatty acids

24. The method of claim 23, wherein the cell or tissue is a tumor cell or tissue.

25. The method of claim 24, wherein the tumor cell is a prostate tumor cell.
